(19) United States
(12) Patent Application Publication
(10) Pub. No.: US 2026/0263665 A1
(43) Pub. Date: Sep. 10, 2026

(54) COATED STAPLES COMPRISING SUCCINATE-BASED COPOLYMERS

(71) Applicant: CILAG GMBH INTERNATIONAL, Zug (CH)

(72) Inventors: LULING WANG, Cincinnati, OH (US); KEVIN WALLEN, Cincinnati, OH (US); HANZHEN BAO, Cincinnati, OH (US); NICHOLAS COURTWRIGHT, Cincinnati, OH (US); EMILY A. SCHELLIN, Cincinnati, OH (US); PEYTON HOPSON, Cincinnati, OH (US); JASON M. RECTOR, Maineville, OH (US); JASON L. HARRIS, Lebanon, OH (US); FREDERICK E. SHELTON, IV, Hillsboro, OH (US)

(73) Assignee: CILAG GMBH INTERNATIONAL, Zug (CH)

(21) Appl. No.: 19/557,997

(22) Filed: Mar. 5, 2026

Related U.S. Application Data
(60) Provisional application No. 63/768,608, filed on Mar. 7, 2025.

(51) Int. Cl. A61L 31/10; A61B 17/064; A61B 17/072; A61L 31/02

(52) U.S. Cl. CPC A61L 31/10; A61B 17/0644; A61B 17/07207; A61L 31/022; A61B 2017/07264; A61B 2017/07271

(57) ABSTRACT

The disclosed technology includes a coated implant (for example, a coated staple) including an implant body (for example, a staple) and a coating succinate-based polymers, wherein the coating is disposed on at least a portion of a surface of the implant body such that the coating delays degradation of the at least one portion of the implant body in vivo.

### CROSS-REFERENCE TO RELATED APPLICATIONS

This application claims the benefit of U.S. Provisional Application Ser. No. 63/768,608 filed Mar. 7, 2025, which is related to U.S. Provisional Application Ser. No. 63/768,629, titled “Method for Controlled Metal Alloy Absorption for Implantable Devices”, filed Mar. 7, 2025, Attorney Docket No. END9548USPSP2_264142.118, the entire contents of which are hereby incorporated by reference in their entirety.

### FIELD

The present invention relates to surgical implants with coatings comprising succinate-based polymers.

### BACKGROUND

Surgical staplers are used in surgical procedures to close openings in tissue, blood vessels, ducts, shunts, or other objects or body parts involved in the particular procedure. The openings can be naturally occurring, such as passageways in blood vessels or an internal organ like the stomach, or they can be formed by the surgeon during a surgical procedure, such as by puncturing tissue or blood vessels to form a bypass or an anastomosis, or by cutting tissue during a stapling procedure.

Most staplers have a handle (some of which are directly user operable, others of which are operable by a user via a robotic interface) with an elongate shaft extending from the handle and having a pair of movable opposed jaws formed on an end thereof for holding and forming staples therebetween. The staples are typically contained in a staple cartridge, which can house multiple rows of staples and is often disposed in one of the two jaws for ejection of the staples to the surgical site. In use, the jaws are positioned so that the object to be stapled is disposed between the jaws, and staples are ejected and formed when the jaws are closed, and the device is actuated. Some staplers include a knife configured to travel between rows of staples in the staple cartridge to longitudinally cut and/or open the stapled tissue between the stapled rows.

Staples or other surgical implants made of absorbable materials degrade in the body over time. While it is important for the staples or other surgical implants to be absorbable to avoid a new surgery to remove the staples or other surgical implants, it is also important to select material that does not absorb too quickly to ensure the staples or other surgical implants retain their shape and strength while a patient heals following a surgery.

Currently, temporary implants (i.e., absorbable implants) consist of three (3) primary classes of materials: polymer, metals, and ceramics. It is expected that during the healing process, for both hard and soft tissue, that the mechanical stiffness/strength of the temporary implants slowly decreases while the surrounding tissue gains mechanical strength/stiffness above a minimum threshold. For polymer-based temporary implants, the microstructure and chemistry can be tailored to control the degradation rate in different physiological environments. While this is a general advantage as the polymer can be tailored to the application, there are limitations due to the inherent mechanical properties of polymers. As such, metal temporary implants offer an advantage with generally higher mechanical properties. Magnesium and its alloys have been studied as temporary implants due to excellent biocompatibility; and magnesium ion facilitation of tissue healing and metabolism cofactor for many enzymes. Another class of materials is ceramics most often associated with calcium phosphates that may be absorbed into the body. These are often used in combination with absorbable polymers for orthopedic applications.

Of particular interest is magnesium alloys given the ability to extrude, machine, etc., into a finished form for implantation. The limiting factor for magnesium in absorbable implants is the high corrosion rate in the physiological environment. This is further complicated as the rate can be accelerated at conditions deviating from a neutral pH condition. This potential uncertainty in the local physiological environment can limit the therapeutic applications of magnesium alloy implants. One mechanism to minimize this impact is coating the magnesium implant with a material to reduce the corrosion sensitivity. These include absorbable polyesters, silane, and calcium phosphate coatings. While these coatings can minimize the rate of magnesium alloy corrosion, these coatings generally degrade through bulk degradation. In the case of polyesters, the localized acid generation during degradation may accelerate the magnesium alloy corrosion in physiological environments.

While Mg corrosion can be designed to follow the healing curve in most cases, the localized differential microstructure can impact the effective loss in mechanical properties as a function of corrosion. This is due to localized stress that can be relieved during the absorption/corrosion process. The uncertainty can be mitigated through design (e.g., volumetric increase in load-bearing geometric features, etc.) or specific post-processing.

As such, the overall limitations of magnesium alloys exist, including potential premature implant fracture, implant feature resolution, sensitivities to both acute and static physiological environments, reactivity of Mg Alloy in nature compared with other implantable alloys such as Ti64, etc. These limitations result in inefficiencies and reduced scope for magnesium alloys as absorbable implants. As such, there is a need to address these limitations.

### SUMMARY

There is provided, in accordance with an example of the present invention, a coated staple comprising:
a staple; and
a coating comprising a copolymer comprising a residue that is derived from a succinate-based monomer,
   wherein the coating is disposed on at least a portion of a surface of the staple, and the coating has an elongation at break (ε_{break}) of at least about 11%.

There is provided, in accordance with an example of the present invention, a coated staple comprising:
a staple comprised substantially of magnesium; and
a coating comprising a copolymer comprising a residue that is derived from a succinate-based monomer,
   wherein the coating is disposed on at least a portion of a surface of the staple, and the coating has an elongation at break (ε_{break}) of at least about 11%.

For example, the coating can cover the entire staple except for the cut edges of the tips.

There is provided, in accordance with an example of the present invention, a coated staple comprising:
a staple comprised substantially of magnesium; and
a coating comprising a copolymer comprising a residue that is derived from a succinate-based monomer,
   wherein the coating is disposed on at least a portion of a surface of the staple, and the coating has a glass transition temperature (Tg) of about −20° C. to about 10° C.

There is provided, in accordance with an example of the present invention, a coated staple comprising:
a staple 300 comprised substantially of magnesium; and
a coating comprising a copolymer comprising a residue that is derived from a succinate-based monomer,
   wherein the coating is disposed on at least a portion of a surface of the staple, wherein when the coating has a thickness of about 2 μm to about 22 μm, the water uptake of the coating is less than 1.9% by weight, or when the coating has a thickness of about 10 μm to about 22 μm, the water uptake of the coating is about 1.9% to about 3.3% by weight.

There is provided, in accordance with an example of the present invention, a method of making the coated staple described above, comprising:
coating at least a portion of a surface of the staple with the copolymer comprising a
structural unit derived from a succinate monomer, wherein coating is conducted via electrospinning, electrospray, dip coating, thermal spray, screen printing, an extrusion process comprising pultrusion, direct deposition, laser sintering, overmolding, spray coating, chemical vapor deposition, reel-to-reel coating, or combinations thereof.

There is provided, in accordance with an example of the present invention, a surgical device, comprising:
a cartridge body, comprising:
a deck; and
a staple cavity defined in the deck, the staple cavity comprising the coated staple described above.

### BRIEF DESCRIPTION OF THE DRAWINGS

This invention will be more fully understood from the following detailed description taken in conjunction with the accompanying drawings, in which:

FIG. 1 is a perspective view of one exemplary embodiment of a conventional surgical stapling and severing instrument.

FIG. 2A is a perspective view of a distal end of the staple cartridge for use with the surgical stapling and severing instrument;

FIG. 2B is a perspective view of a projection extending from the staple cartridge;

FIG. 3 is a side view of a staple in an unfired (pre-deployed) configuration that can be disposed within the staple cartridge of the surgical cartridge assembly of FIG. 2A;

FIG. 4 is a perspective view of a knife and firing bar (“E-beam”) of the surgical stapling and severing instrument of FIG. 1;

FIG. 5 is a perspective view of a wedge sled of a staple cartridge of the surgical stapling and severing instrument of FIG. 1;

FIG. 6A is a longitudinal cross-sectional view of an exemplary surgical cartridge assembly having a compressible non-fibrous adjunct attached to a top or deck surface of a staple cartridge;

FIG. 6B is a longitudinal cross-sectional view of a surgical end effector having an anvil pivotably coupled to an elongate channel and the surgical cartridge assembly of FIG. 6A disposed within and coupled to the elongate channel, showing the anvil in a closed position without any tissue between the anvil and the adjunct;

FIG. 7 is a partial-schematic illustrating the adjunct of FIGS. 6A-6B in a tissue deployed condition;

FIGS. 8A-8B show top plan views of two staples: FIG. 8A shows a three-dimensional shape of staple, and FIG. 8B shows a two-dimensional shape of staple;

FIGS. 9A-9E are images of the staples coated with low molecular weight poly(L-lactic acid) (LMW PLLA) (FIG. 9A), high molecular weight poly(L-lactic acid) (HMW PLLA) (FIG. 9B), a succinate-based polymer (CSS polymer) (FIG. 9C), polycaprolactone (PCL) (FIG. 9D), and polyester polyurethane (PEU) (FIG. 9E). The thickness of the coating is about 6 μm in each of FIG. 9A-9E;

FIGS. 10A-10E show representative images of the staples in the staple aging test for staples coated with PEU1 (Cyprex PEU V8F6-9010 from Viamer) at 22° C. (FIG. 10A), PEU1 at 38° C. (FIG. 10B), CSS polymer (a succinate-based polymer, 25% CSS) at 38° C. (FIG. 10C), PCL (Resomer C 212 from Evonik) at 38° C. (FIG. 10D), and PEU2 (Cyprex PEU V8 from Viamer) at 38° C. (FIG. 10E).

### DETAILED DESCRIPTION

The following detailed description should be read with reference to the drawings, in which like elements in different drawings are identically numbered. The drawings, which are not necessarily to scale, depict selected embodiments and are not intended to limit the scope of the invention. The detailed description illustrates by way of example, not by way of limitation, the principles of the invention. This description will clearly enable one skilled in the art to make and use the invention, and describes several embodiments, adaptations, variations, alternatives and uses of the invention, including what is presently believed to be the best mode of carrying out the invention.

As used herein, the terms “about” or “approximately” for any numerical values or ranges indicate a suitable dimensional tolerance that allows the part or collection of components to function for its intended purpose as described herein. More specifically, “about” or “approximately” may refer to the range of values±10% of the recited value, e.g., “about 90%” may refer to the range of values from 81% to 99%.

As used herein, the term “alkenyl” means a straight or branched alkyl group having one or more double carbon-carbon bonds and 2-20 carbon atoms, including, but not limited to, ethenyl, 1-propenyl, 2-propenyl, 2-methyl-1-propenyl, 1-butenyl, 2-butenyl, and the like. In some embodiments, the alkenyl chain is from 2 to 10 carbon atoms in length, from 2 to 8 carbon atoms in length, from 2 to 6 carbon atoms in length, or from 2 to 4 carbon atoms in length.

The terms “alkoxy”, “phenyloxy”, “benzoxy” and “pyrimidinyloxy” refer to an alkyl group, phenyl group, benzyl group, or pyrimidinyl group, respectively, each optionally substituted, that is bonded through an oxygen atom. For example, the term “alkoxy” means a straight or branched —O-alkyl group of 1 to 20 carbon atoms, including, but not limited to, methoxy, ethoxy, n-propoxy, isopropoxy, t-butoxy, and the like. In some embodiments, the alkoxy chain is from 1 to 10 carbon atoms in length, from 1 to 8 carbon atoms in length, from 1 to 6 carbon atoms in length, from 1 to 4 carbon atoms in length, from 2 to 10 carbon atoms in length, from 2 to 8 carbon atoms in length, from 2 to 6 carbon atoms in length, or from 2 to 4 carbon atoms in length.

As used herein, the term “alkyl” means a saturated hydrocarbon group which is straight-chained or branched. An alkyl group can contain from 1 to 20, from 2 to 20, from 1 to 10, from 2 to 10, from 1 to 8, from 2 to 8, from 1 to 6, from 2 to 6, from 1 to 4, from 2 to 4, from 1 to 3, or 2 or 3 carbon atoms. Examples of alkyl groups include, but are not limited to, methyl (Me), ethyl (Et), propyl (e.g., n-propyl and isopropyl), butyl (e.g., n-butyl, t-butyl, isobutyl), pentyl (e.g., n-pentyl, isopentyl, neopentyl), hexyl, isohexyl, heptyl, 4,4-dimethylpentyl, octyl, 2,2,4-trimethylpentyl, nonyl, decyl, undecyl, dodecyl, 2-methyl-1-propyl, 2-methyl-2-propyl, 2-methyl-1-butyl, 3-methyl-1-butyl, 2-methyl-3-butyl, 2-methyl-1-pentyl, 2,2-dimethyl-1-propyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, and the like.

As used herein, the term “alkylene” or “alkylenyl” means a divalent alkyl linking group. An example of an alkylene (or alkylenyl) is methylene or methylenyl (—CH₂—).

As used herein, the term “alkynyl” means a straight or branched alkyl group having one or more triple carbon-carbon bonds and 2-20 carbon atoms, including, but not limited to, acetylene, 1-propylene, 2-propylene, and the like. In some embodiments, the alkynyl chain is 2 to 10 carbon atoms in length, from 2 to 8 carbon atoms in length, from 2 to 6 carbon atoms in length, or from 2 to 4 carbon atoms in length.

As used herein, the term “amino” means-NH₂.

As used herein, the term “aryl” means a monocyclic, bicyclic, or polycyclic (e.g., having 2, 3 or 4 fused rings) aromatic hydrocarbons optionally comprising heteroatoms. In some embodiments, aryl groups have from 6 to 20 carbon atoms or from 6 to 10 carbon atoms. Examples of aryl groups include, but are not limited to, phenyl, naphthyl, anthracenyl, phenanthrenyl, indanyl, indenyl, tetrahydronaphthyl, and the like. In some embodiments, aryl groups include heteroaryl groups defined in other embodiments. In some embodiments, aryl groups have heteroatoms. In some embodiments, aryl groups have heteroatoms selected from the group consisting of N, S, O, and P atoms.

As used herein, the term “cycloalkyl” means non-aromatic cyclic hydrocarbons including cyclized alkyl, alkenyl, and alkynyl groups that contain up to 20 ring-forming carbon atoms. Cycloalkyl groups can include mono- or polycyclic ring systems such as fused ring systems, bridged ring systems, and spiro ring systems. In some embodiments, polycyclic ring systems include 2, 3, or 4 fused rings. A cycloalkyl group can contain from 3 to 15, from 3 to 10, from 3 to 8, from 3 to 6, from 4 to 6, from 3 to 5, or 5 or 6 ring-forming carbon atoms. Ring-forming carbon atoms of a cycloalkyl group can be optionally substituted by oxo or sulfido. Examples of cycloalkyl groups include, but are not limited to, cyclopropyl, cyclobutyl, cyclopentyl, cyclohexyl, cycloheptyl, cyclooctyl, cyclononyl, cyclopentenyl, cyclohexenyl, cyclohexadienyl, cycloheptatrienyl, norbornyl, norpinyl, norcarnyl, adamantyl, and the like. Also included in the definition of cycloalkyl are moieties that have one or more aromatic rings fused (having a bond in common with) to the cycloalkyl ring, for example, benzo or thienyl derivatives of pentane, pentene, hexane, and the like (e.g., 2,3-dihydro-1H-indene-1-yl, or 1H-inden-2(3H)-one-1-yl).

As used herein, the term “heteroaryl” means an aromatic heterocycle having up to 20 ring-forming atoms (e.g., C) and having at least one heteroatom ring member (ring-forming atom) such as sulfur, oxygen, or nitrogen. In some embodiments, the heteroaryl group has at least one or more heteroatom ring-forming atoms, each of which are, independently, sulfur, oxygen, or nitrogen. In some embodiments, the heteroaryl group has from 3 to 20 ring-forming atoms, from 3 to 10 ring-forming atoms, from 3 to 6 ring-forming atoms, or from 3 to 5 ring-forming atoms. In some embodiments, the heteroaryl group contains 2 to 14 carbon atoms, from 2 to 7 carbon atoms, or 5 or 6 carbon atoms. In some embodiments, the heteroaryl group has 1 to 4 heteroatoms, 1 to 3 heteroatoms, or 1 or 2 heteroatoms. Heteroaryl groups include monocyclic and polycyclic (e.g., having 2, 3 or 4 fused rings) systems. Examples of heteroaryl groups include, but are not limited to, pyridyl, pyrimidinyl, pyrazinyl, pyridazinyl, triazinyl, furyl, quinolyl, isoquinolyl, thienyl, imidazolyl, thiazolyl, indolyl (such as indol-3-yl), pyrroyl, oxazolyl, benzofuryl, benzothienyl, benzthiazolyl, isoxazolyl, pyrazolyl, triazolyl, tetrazolyl, indazolyl, 1,2,4-thiadiazolyl, isothiazolyl, benzothienyl, purinyl, carbazolyl, benzimidazolyl, indolinyl, pyranyl, oxadiazolyl, isoxazolyl, triazolyl, thianthrenyl, pyrazolyl, indolizinyl, isoindolyl, isobenzofuranyl, benzoxazolyl, xanthenyl, 2H-pyrrolyl, pyrrolyl, 3H-indolyl, 4H-quinolizinyl, phthalazinyl, naphthyridinyl, quinazolinyl, phenanthridinyl, acridinyl, perimidinyl, phenanthrolinyl, phenazinyl, isothiazolyl, phenothiazinyl, isoxazolyl, furanyl, phenoxazinyl groups, and the like. Suitable heteroaryl groups include 1,2,3-triazole, 1,2,4-triazole, 5-amino-1,2,4-triazole, imidazole, oxazole, isoxazole, 1,2,3-oxadiazole, 1,2,4-oxadiazole, 3-amino-1,2,4-oxadiazole, 1,2,5-oxadiazole, 1,3,4-oxadiazole, pyridine, and 2-aminopyridine.

As used herein, the term “heterocycle” or “heterocyclic ring” means a 5- to 7-membered mono- or bicyclic or 7- to 10-membered bicyclic heterocyclic ring system any ring of which may be saturated or unsaturated, and which consists of carbon atoms and from one to three heteroatoms chosen from N, O and S, and wherein the N and S heteroatoms may optionally be oxidized, and the N heteroatom may optionally be quaternized, and including any bicyclic group in which any of the above-defined heterocyclic rings is fused to a benzene ring. Particularly useful are rings containing one oxygen or sulfur, one to three nitrogen atoms, or one oxygen or sulfur combined with one or two nitrogen atoms. The heterocyclic ring may be attached at any heteroatom or carbon atom which results in the creation of a stable structure. Examples of heterocyclic groups include, but are not limited to, piperidinyl, piperazinyl, 2-oxopiperazinyl, 2-oxopiperidinyl, 2-oxopyrrolodinyl, 2-oxoazepinyl, azepinyl, pyrrolyl, 4-piperidonyl, pyrrolidinyl, pyrazolyl, pyrazolidinyl, imidazolyl, imidazolinyl, imidazolidinyl, pyridyl, pyrazinyl, pyrimidinyl, pyridazinyl, oxazolyl, oxazolidinyl, isoxazolyl, isoxazolidinyl, morpholinyl, thiazolyl, thiazolidinyl, isothiazolyl, quinuclidinyl, isothiazolidinyl, indolyl, quinolinyl, isoquinolinyl, benzimidazolyl, thiadiazoyl, benzopyranyl, benzothiazolyl, benzoxazolyl, furyl, tetrahydrofuryl, tetrahydropyranyl, thienyl, benzothienyl, thiamorpholinyl, thiamorpholinyl sulfoxide, thiamorpholinyl sulfone, and oxadiazolyl. Morpholino is the same as morpholinyl.

As used herein, the term “heterocycloalkyl” means non-aromatic heterocycles having up to 20 ring-forming atoms including cyclized alkyl, alkenyl, and alkynyl groups, where one or more of the ring-forming carbon atoms is replaced by a heteroatom such as an O, N, or S atom. Heterocycloalkyl groups can be mono or polycyclic (e.g., fused, bridged, or spiro systems). In some embodiments, the heterocycloalkyl group has from 1 to 20 carbon atoms, or from 3 to 20 carbon atoms. In some embodiments, the heterocycloalkyl group contains 3 to 14 ring-forming atoms, 3 to 7 ring-forming atoms, or 5 or 6 ring-forming atoms. In some embodiments, the heterocycloalkyl group has 1 to 4 heteroatoms, 1 to 3 heteroatoms, or 1 or 2 heteroatoms. In some embodiments, the heterocycloalkyl group contains 0 to 3 double bonds. In some embodiments, the heterocycloalkyl group contains 0 to 2 triple bonds. Examples of heterocycloalkyl groups include, but are not limited to, morpholino, thiomorpholino, piperazinyl, tetrahydrofuranyl, tetrahydrothienyl, 2,3-dihydrobenzofuryl, 1,3-benzodioxole, benzo-1,4-dioxane, piperidinyl, pyrrolidinyl, isoxazolidinyl, oxazolidinyl, isothiazolidinyl, pyrazolidinyl, thiazolidinyl, imidazolidinyl, pyrrolidin-2-one-3-yl, and the like. In addition, ring-forming carbon atoms and heteroatoms of a heterocycloalkyl group can be optionally substituted by oxo or sulfido. For example, a ring-forming S atom can be substituted by 1 or 2 oxo (form a S(O) or S(O)₂). For another example, a ring-forming C atom can be substituted by oxo (form carbonyl). Also included in the definition of heterocycloalkyl are moieties that have one or more aromatic rings fused (having a bond in common with) to the nonaromatic heterocyclic ring including, but not limited to, pyridinyl, thiophenyl, phthalimidyl, naphthalimidyl, and benzo derivatives of heterocycles such as indolene, isoindolene, 4,5,6,7-tetrahydrothieno[2,3-c]pyridine-5-yl, 5,6-dihydrothieno[2,3-c]pyridin-7 (4H)-one-5-yl, isoindolin-1-one-3-yl, and 3,4-dihydroisoquinolin-1 (2H)-one-3yl groups. Ring-forming carbon atoms and heteroatoms of the heterocycloalkyl group can be optionally substituted by oxo or sulfido.

As used herein, the term “halo” means halogen groups including, but not limited to fluoro, chloro, bromo, and iodo.

As used herein, the term “elongation at break” or “ε_{break}” refers to the additional extent to which a material can be stretched or elongated before it fractures. It is expressed as a percentage (%) of the original length of the material.

As used herein, the term “Young's modulus” refers to a material property that measures the stiffness of a solid material. It is defined as the ratio of tensile stress (force per unit area) to tensile strain (proportional deformation) in the linear elasticity regime of a material. Mathematically, it is expressed as:

$$E = σ / ε$$

where E is Young's modulus, σ is the tensile stress, and ε is the tensile strain. Young's modulus is typically measured in pascals (Pa) and provides insight into the material's ability to deform elastically (i.e., non-permanently) when a force is applied. A higher Young's modulus indicates a stiffer material, while a lower Young's modulus indicates a more flexible material.

As used herein, the term “glass transition temperature” (Tg) refers to the temperature at which a polymer coating transitions from a hard, glassy state to a soft, rubbery state. This temperature is a critical thermal property of polymers and indicates the point at which the polymer chains gain sufficient mobility to move past one another, resulting in a significant change in mechanical properties. The glass transition temperature affects their flexibility, hardness, and overall durability.

As used herein, the term “water uptake” refers to the amount of water absorbed by a polymer when it is exposed to a humid environment or immersed in water. This property is expressed as a percentage of the polymer's original weight. Water uptake evaluates the performance and durability of polymers, especially in applications where the material will be exposed to moisture. High water uptake can lead to swelling, changes in mechanical properties, and potential degradation of the polymer. Water uptake is usually determined through standardized testing methods, where the polymer sample is weighed before and after exposure to water, and the increase in weight is used to calculate the percentage of water absorbed.

As used herein, the term “succinic acid” refers to H—OC(O)—(CH₂—CH₂)—C(O)O—H.

As used herein, the term “succinate” refers to the anion form of succinic acid, e.g., [O]—C(O)—(CH₂—CH₂)—C(O)—[O⁻].

As used herein, the term “a succinate-based monomer” or “CSS monomer” refers to a monomer that contains succinic acid or its derivatives as a fundamental component of its molecular structure. In some embodiments, the term “a succinate-based monomer” or “CSS monomer” refers to a monomer that includes at least one structural segment that is *—C(O)—(CH₂—CH₂)—C(O)—* (* indicates a point of attachment). The term “succinate-based” and “CSS” are used interchangeably.

As used herein, the term “residue” refers to a single molecular unit within a polymer.

As used herein, the term “succinate-based polymer”, “succinate-based copolymer”, or “CSS polymer” refers to a type of polymer that incorporates succinic acid or its derivatives as a monomer in its backbone structure. In some embodiments, the term “succinate-based polymer”, “succinate-based copolymer”, or “CSS polymer” refers to a copolymer comprising a residue that is a succinate-based monomer. In some embodiments, “succinate-based polymer”, “succinate-based copolymer”, or “CSS polymer” refers to a copolymer comprising one or more structural units derived from succinic acid.

As used herein, “% CSS” refers to the CSS stoichiometry represented by the mole percentage of the residue that is a succinate-based monomer in the CSS polymer. For example, a succinate-based polymer comprising 15% CSS means that the polymer comprises 15 mole % of CSS monomer or 1 mole of the succinate-based polymer comprises 0.15 stoichiometry mole of CSS monomers.

As used herein, “comprise substantially of” or “substantially composed of” refers to comprising at least about 90% by weight.

As used herein, “significantly” refers to an amount in which a particular component has the greatest compositional quantity among a plurality of components in a composition. In some embodiments, “comprise significantly” or “significantly composed of” refers to comprising a metal having the greatest compositional quantity among a plurality of other components in the alloy.

As used herein, “comprise principally” or “principally composed of” refers to comprising more than about 50% by weight.

As used herein, “suitable substituent”, “substituent”, “optional substituent”, or the substituent for any optionally substituted group means a group that does not nullify the synthetic or pharmaceutical utility of the compounds described herein or the intermediates useful for preparing them. Examples of “suitable substituent”, “substituent”, “optional substituent”, or the substituent for any optionally substituted group include, but are not limited to: C₁-C₆alkyl, C₁-C₆alkenyl, C₁-C₆alkynyl, C₁-C₆alkoxy, phenyl, C₃-C₅heteroaryl, C₃-C₁₀cycloalkyl, C₃-C₁₀heterocycloalkyl, C₅-C₆aryloxy, —CN, —OH, oxo, halo, haloalkyl, —NO₂, —CO₂H, —NH₂, —NH(C₁-C₆alkyl), —N(C₁-C₆alkyl)₂, —NH(phenyl), —N(phenyl)₂, —CHO, —CO(C₁-C₆alkyl), —CO(phenyl), —CO₂(C₁-C₆alkyl), and —CO₂ (phenyl). One of skill in the art can readily choose a suitable substituent based on the stability and pharmacological and synthetic activity of the compounds described herein.

As used herein, the “thickness” of a coating means the mean thickness. Due to non-uniform concentricity of the wire or coating process, the actual thickness of the coating may vary following the normal distribution. In some embodiments, the coating having a thickness of about 6 μm means the mean thickness is about 6 μm. The coating having a thickness of about 19 μm means the mean thickness is about 19 μm, with a standard deviation of about 6 μm. As used herein, the “minimum thickness” of a coating refers to the threshold thickness (about 1.65*standard deviation away from the mean) for the lowest 10% of coating thickness within a normal distribution. For example, when the coating having a thickness of about 6 μm with a standard deviation of about 2 μm, the minimum thickness is about 2.7 μm (2.7 μm is the threshold of the lowest 10% of coating thickness within a normal distribution).

Coatings can prolong the duration that an absorbable surgical implant such as a metal or metal alloy staple will last in the body, thereby extending the time the body can heal before being relied upon to deliver full strength at the cut line. For example, the coating can cover the entire staple except for the cut edges of the tips. When the coating is damaged, the staples start to degrade at the damage location quickly. In particular, when firing staples out of a staple cartridge into pockets of an anvil of a stapler, their coatings can be scrapped off at the tips and sides. Thus, there is a need to prevent coating damage to the tips and sides of staples or other surgical implants.

A person skilled in the art will appreciate that the coated implant or coated staple can include only some of these features and/or it can include a variety of other features known in the art. Coated implants described herein are merely intended to represent certain exemplary examples.

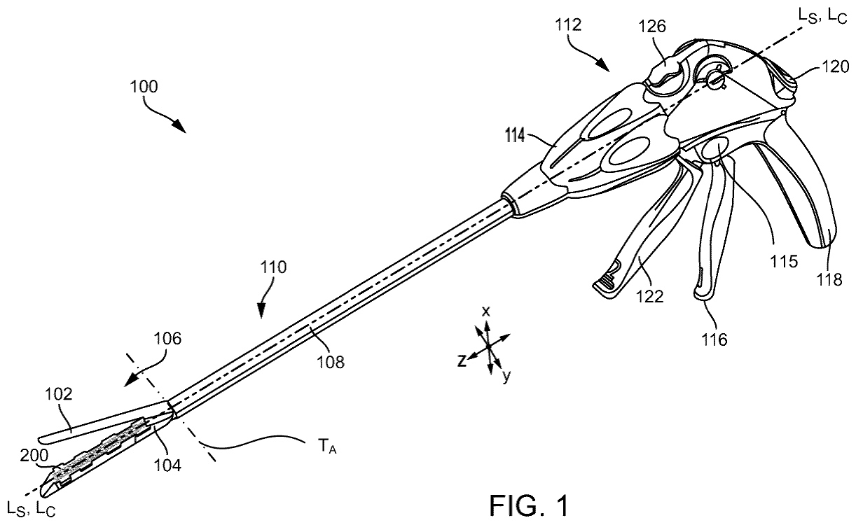

FIG. 1 illustrates an exemplary surgical stapling and severing device 100 suitable for use with an implantable adjunct. The illustrated surgical stapling and severing device 100 includes end effector 106 having an anvil 102 that is pivotably coupled to an elongate channel 104. As a result, the staple applying assembly 106 can move between an open position, as shown in FIG. 1, and a closed position in which the anvil 102 is positioned adjacent to the elongate channel 104 to engage tissue therebetween. The end effector 106 can be attached at its proximal end to an elongate shaft 108, forming an implement portion 110. When the end effector 106 is closed, or at least substantially closed (e.g., the anvil 102 moves from the open position in FIG. 1 toward the elongate channel) the implement portion 110 can present a sufficiently small cross-section suitable for inserting the end effector 106 through a trocar. While the device 100 is configured to staple and sever tissue, surgical devices configured to staple but not sever tissue are also contemplated herein.

In various instances, the end effector 106 can be manipulated by a handle 112 connected to the elongate shaft 108. The handle 112 can include user controls such as a rotation knob 114 that rotates the elongate shaft 108 and the end effector 106 about a longitudinal axis (Ls) of the elongate shaft 108 and an articulation control 115 that can articulate the end effector 106 about an articulate axis (TA) that is substantially transverse to the longitudinal axis (Ls) of the elongate shaft 108. Further controls include a closure trigger 116 which can pivot relative to a pistol grip 118 to close the end effector 106. A closure release button 120 can be outwardly presented on the handle 112 when the closure trigger 116 is clamped such that the closure release button 120 can be depressed to unclamp the closure trigger 116 and open the end effector 106, for example. Handle 112 may also take the form of an interface for connection to a surgical robot.

In some examples, a firing trigger 122, which can pivot relative to the closure trigger 116, can cause the end effector 106 to simultaneously sever and staple tissue clamped therein. The firing trigger 122 may be a powered, require force from a user to engage, or some combination thereof. A manual firing release lever 126 can allow the firing system to be retracted before full firing travel has been completed, if desired, and, in addition, the firing release lever 126 can allow a surgeon, or other clinician, to retract the firing system in the event that the firing system binds and/or fails.

Additional details on the surgical stapling and severing device 100 and other surgical stapling and severing devices suitable for use with the present disclosure are described, for example, in U.S. Pat. No. 9,332,984 and in U.S. Patent Publication No. 2009/0090763, the disclosures of which are incorporated herein by reference in their entireties. Further, the surgical stapling and severing device need not include a handle, but instead can have a housing that is configured to couple to a surgical robot, for example, as described in U.S. Patent Publication No. 2019/0059889, the disclosure of which is incorporated herein by reference in its entirety.

Alternatively, a powered articulate surgical stapling and severing instrument can be used. A suitable powered articulate surgical stapling and severing instrument that can be used according to the present disclosure is described in U.S. Pat. No. 11,723,662, the disclosure of which is incorporated herein by reference in its entirety.

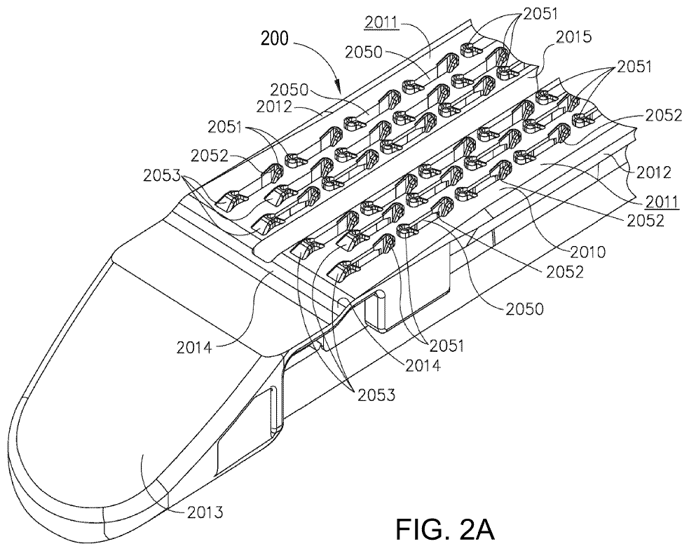

As further shown in FIG. 1, a staple cartridge 200 can be utilized with the instrument 100. In use, the staple cartridge 200 is placed within and coupled to the elongate channel 104. While the staple cartridge 200 can have a variety of configurations, in this illustrated example, the staple cartridge 200, which is shown in more detail in FIGS. 2A-2B. As shown in FIG. 2A, the cartridge body 2010 can further comprise a plurality of projections 2051 extending from the deck surface 2011. Projections 2051 can be configured to engage tissue positioned intermediate the anvil 102 and the cartridge 200 and control the movement of the tissue relative to the cartridge 200. Tissue can move relative to the cartridge 200 in various instances. In at least one instance, tissue can flow relative to the cartridge 200 when the anvil 102 is moved between an open position and a closed position in which the tissue is squeezed between the anvil 102 and the cartridge 200. In such instances, the tissue may flow laterally toward the longitudinal sides 2012, distally toward the distal end 2013, and/or proximally toward the proximal end 2016. In at least one other instance, tissue can flow relative to the cartridge 200 when the cutting edge is advanced distally through the tissue captured between the anvil 102 and the cartridge 200. In such instances, tissue may flow laterally, distally, and/or proximally, but it primarily flows distally due to the distal movement of the cutting edge. In various instances, projections 2051 can be configured to limit or prevent the flow of the tissue relative to the staple cartridge. Projections 2051 can be positioned at the proximal end and/or the distal end of the staple cavities 2050. In various instances, each projection 2051 can comprise a cuff extending around an end of a staple cavity 2050. In certain instances, each projection 2051 can comprise an arcuate ridge extending around an end of a staple cavity 2050.

As shown in FIG. 2A, the cartridge body 2010 can comprise a sloped transition 2014 extending between the distal tip of the cartridge body 2010 and the deck surface 2011. The sloped transition 2014 can facilitate the movement of the cartridge 200 relative to the tissue when positioning the cartridge 200 and the anvil 102 within a surgical site. In such instances, the tissue can slide over the sloped surface 2014. In various instances, the sloped surface 2014 can comprise a radiused surface. In various instances, the sloped surface 2014 can comprise an angled surface. In certain instances, the sloped surface 2014 can comprise a concave surface and/or a convex surface. In at least one instance, as illustrated in FIG. 2A, the sloped surface 2014 can comprise a distal concave surface which transitions into a flat, angled surface which transitions into a proximal convex surface, for example.

The staple cavities 2050 defined in the cartridge body 2010 can be arranged in longitudinal rows. For instance, three longitudinal rows of staple cavities 2050 can be arranged on a first side of the longitudinal slot 2015 and three longitudinal rows of staple cavities 2050 can be arranged on a second side of the longitudinal slot 2105. Each longitudinal row can include a distal-most staple cavity 2050 adjacent to the distal end 2013 and a proximal-most staple cavity 2050 adjacent to the proximal end 2016. In various instances, the cartridge body 2010 can further comprise projections 2053 extending from the deck surface 2011. The projections 2053 can be positioned at the distal ends of the distal-most staple cavities 2050. Each projection 2053 can comprise a distal sloped surface, for example, configured to facilitate the insertion of the tissue between the staple cartridge 200 and the anvil 102. In various instances, the distal-most cavities 2050 can each include a projection 2053 positioned at the distal end thereof and a projection 2051 positioned at a proximal end thereof.

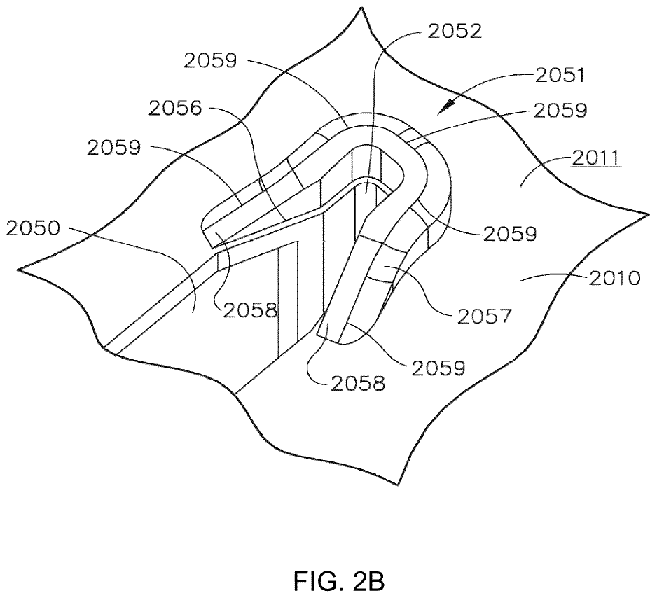

Each projection 2051 and/or projection 2053 can be configured to support at least a portion of a staple removably stored in a staple cavity 2050. In various instances, each projection 2051 can extend an endwall 2052 of the staple cavity 2050 above the deck 2011. In certain instances, referring generally to FIG. 3, a staple positioned within the staple cavity 2050 can include a base 1002, a first leg 1004 extending from the base at a first angle, and a second leg 1006 extending from the base at a second angle. The first leg 1004 can be in contact with a first endwall 2052 (FIG. 2A) of a staple cavity 2050 and the second leg 1006 can be in contact with a second endwall 2052 of the staple cavity. In certain instances, the distance, or spread, between the first leg and the second leg of the staple can be wider than the distance between the endwalls 2052 such that, when the staple is positioned within the staple cavity 2050, the legs are biased inwardly by the endwalls 2052. When the staple is stored within the staple cavity 2050 in its unfired, or unlifted, position, the tips of the staple legs may be positioned within the projections 2051. In such instances, the projections 2051 can support and protect the tips of the staple legs above the deck 2011. In some instances, the tips of the staple legs may be positioned below the projections 2051 when the staple is in its unfired position and, thus, the projections 2051 may not support the staple legs when the staple is in its unfired position. When such a staple is fired, or lifted out of the staple cavity 2050, the staple legs may then come into contact with and be supported by the projections 2051. In any event, the projections 2051 can continue to support the staple legs as the staple is deployed until the staple has been sufficiently fired and/or lifted out of the staple cavity 2050 such that the staple legs are no longer in contact with the projections 2051. Projections 2053 can perform in a similar manner as that described in connection with projections 2051.

In various instances, further to the above, a projection 2051 can extend a staple cavity 2050 above the deck 2011 of the cartridge body 2010. In certain instances, the projection 2051 can be configured such that an endwall 2052 of the staple cavity 2050 extends seamlessly into the projection 2051. Stated another way, a seamless surface can be defined between the endwall 2052 and the projection 2051. Such a seamless surface can reduce the possibility of a staple leg which is biased against the endwall 2052 and the projection 2051 from contacting an edge or step defined within the staple cavity 2050 and/or digging into the sidewall of the staple cavity 2050. FIG. 2B illustrates a step 2056 defined between the projection 2051 and the endwall 2052. While the step 2056 comprises an outward step and not an inward step, the step 2056 can be eliminated to provide a seamless surface as discussed above. In embodiments where the cartridge body 2010 is formed during an injection molding process, the cartridge 2010 can be formed in a mold cavity defined between two halves of an injection mold. The two halves of the injection mold can contact one another to seal, or at least substantially seal, the mold cavity. The interface between the two mold halves is often referred to as the seal line, or parting line, and, oftentimes, a small ledge or lip is formed in the cartridge body along the seal line. This is especially true when the seal line is used to vent air from the mold cavity during the injection molding process. This ledge or lip is often referred to as ‘flash’. The injection mold can be carefully designed such that the seal line does not produce a ledge or lip in the endwall 2052 and/or the inwardly-facing surface of the projection 2051. In at least one instance, the projection 2051 can comprise an end cuff portion 2057 and a transition portion 2058 extending from opposite ends of the cuff portion to the deck 2011. The seal line between the two mold halves can be selected such that it extends along the top surface of the projection 2051. An exemplary seal line 2059 is depicted in FIG. 2B, although other suitable seal lines could be selected. Additional details on the staple cartridge 200 suitable for use with the present disclosure are described, for example, in U.S. Pat. No. 11,517,315, the disclosure of which is incorporated herein by reference in its entirety.

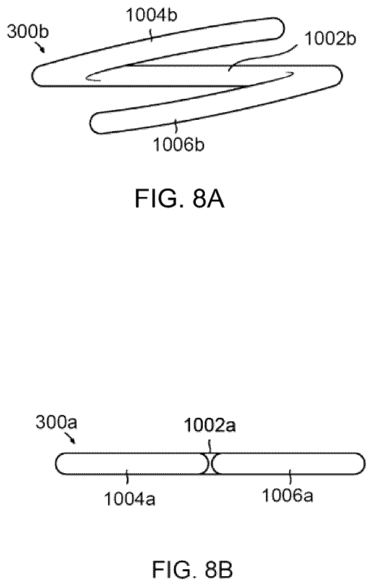

The staples releasably stored in the staple cavities 2050 can have a variety of configurations. An exemplary staple 300a or 300b (shown in FIGS. 8A-8B) that can be releasably stored in each of the staple cavities 2050 in its unfired (pre-deployed) configuration. The illustrated staple 300 includes a crown (base) 1002 and two legs 1004 and 1006 extending from each end of the crown 1002. In this example, the crown 1002 extends in a linear direction and the staple legs 1004 and 1006 have the same unfired height. Further, prior to the staples 300 or coated staples being deployed, the staple crowns 1002 can be supported by staple drivers that are positioned within the staple cartridge 200 and, concurrently, the staple legs 1004 and 1006 can be at least partially contained within the staple cavities 2050. In certain instances, as shown in FIG. 3, the tips 1009 and 1010 of the staple legs 1004 and 1006 can be pointed and sharp which can incise and penetrate tissue. In use, staples 300 can be deformed from an unfired position into a fired position such that the staple legs 1004 and 1006 move through the staple cavities 2050, penetrate tissue positioned between the anvil 102 and the staple cartridge 200, and contact the anvil 102. As the staple legs 1004 and 1006 are deformed against the anvil 102, the legs 1004 and 1006 of each staple 300 can capture a portion of the tissue within each staple 300 and apply a compressive force to the tissue. Further, the legs 1004 and 1006 of each staple 300 can be deformed downwardly toward the crown 1002 of the staple 300 to form a staple entrapment area in which the tissue can be captured therein. In various instances, the staple entrapment area can be defined between the inner surfaces of the deformed legs and the inner surface of the crown of the staple. The size of the entrapment area for a staple can depend on several factors such as the length of the legs, the diameter of the legs, the width of the crown, and/or the extent in which the legs are deformed, for example.

In some examples, all of the staples disposed within the staple cartridge 200 can have the same unfired (pre-deployed) configuration. In other examples, the staples can include at least two groups of staples each having a different unfired (pre-deployed) configuration, e.g., varying in height and/or shape, relative to one another, etc.

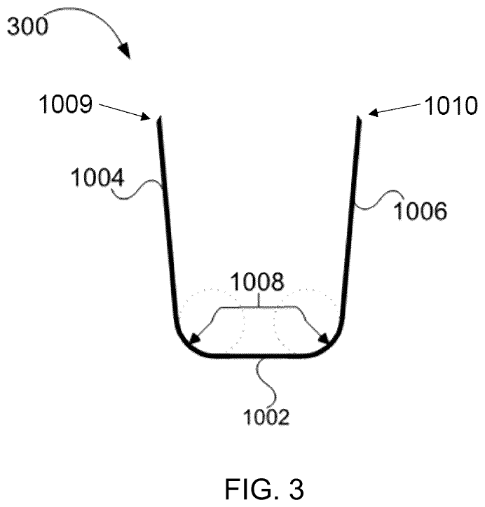

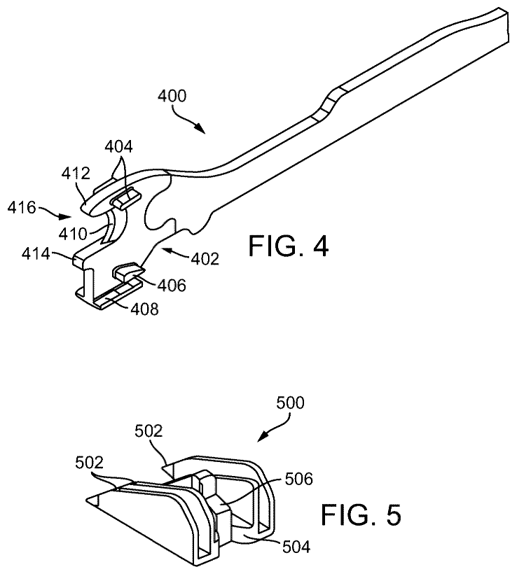

With reference to FIGS. 4 and 5, a firing assembly such as, for example, firing assembly 400, can be utilized with a surgical stapling and severing device, like device 100 in FIG. 1. The firing assembly 400 can be configured to advance a wedge sled 500 having wedges 502 configured to deploy staples from the staple cartridge 200 into tissue captured between an anvil, like anvil 102 in FIG. 1, and a staple cartridge, like staple cartridge 200 in FIG. 1. Furthermore, an E-beam 402 at a distal portion of the firing assembly 400 may fire the staples from the staple cartridge. During firing, the E-beam 402 can also cause the anvil to pivot towards the staple cartridge, and thus move the end effector from the open position towards a closed position. The illustrated E-beam 402 includes a pair of top pins 404, a pair of middle pins 406, which may follow a portion 504 of the wedge sled 500, and a bottom pin or foot 408. The E-beam 402 can also include a sharp cutting edge 410 configured to sever the captured tissue as the firing assembly 400 is advanced distally, and thus towards the distal end of the staple cartridge. In addition, integrally formed and proximally projecting top guide 412 and middle guide 414 bracketing each vertical end of the cutting edge 410 may further define a tissue staging area 416 assisting in guiding tissue to the sharp cutting edge 410 prior to being severed. The middle guide 414 may also serve to engage and fire the staples within the staple cartridge by abutting a stepped central member 506 of the wedge sled 500 that affects staple formation by the end effector 106.

Alternatively, the surgical stapling and severing device 100 further comprises a closure actuator configured to drive a closure system of the surgical stapling and severing device 100 and move the second jaw between an unclamped position and a clamped position. Additional details of a closure actuator suitable for use with the present disclosure are described, for example, in U.S. Pat. No. 11,723,662, the disclosure of which is incorporated herein by reference in its entirety.

Moreover, the second jaw can translate and rotate as it is being moved into its clamped position. In various alternative embodiments, the surgical stapling and severing device 100 comprises a staple cartridge jaw which is movable between an unclamped position and a clamped position relative to an anvil jaw. In any event, the handle comprises a lock configured to releasably hold the closure actuator in its clamped position. The handle further comprises release actuators which, when either one is actuated, unlock the closure actuator such that the end effector can be re-opened. In various alternative embodiments, the handle comprises an electric motor configured to move the closure tube proximally and/or distally when actuated by the clinician. Additional details on second jaw, staple cartridge, and release actuators suitable for use with the present disclosure are described, for example, in U.S. Pat. No. 11,723,662, the disclosure of which is incorporated herein by reference in its entirety.

To deploy staples from the staple cartridge, as discussed above, the sled 500 in FIG. 5 can be moved from the proximal end toward a distal end of the cartridge body, and thus, of the staple cartridge. As the firing assembly 400 in FIG. 4 is advanced, the sled can contact and lift staple drivers within the staple cartridge upwardly within the staple cavities 212, 214. In at least one example, the sled and the staple drivers can each include one or more ramps, or inclined surfaces, which can co-operate to move the staple drivers upwardly from their unfired positions. As the staple drivers are lifted upwardly within their respective staple cavities, the staples are advanced upwardly such that the staples emerge from their staple cavities and penetrate into tissue. In various instances, the sled can move several staples upwardly at the same time as part of a firing sequence. Additional details on the sled suitable for use with the present disclosure are described, for example, in U.S. Pat. Appl. No. 2022/0133305, the disclosure of which is incorporated herein by reference in its entirety.

As indicated above, the stapling device can be used in combination with a compressible adjunct. A person skilled in the art will appreciate that, while adjuncts are shown and described below, the adjuncts disclosed herein can be used with other surgical instruments and need not be coupled to a staple cartridge as described. Further, a person skilled in the art will also appreciate that the staple cartridges need not be replaceable.

In some instances, a surgical stapling instrument can be equipped with a buttress material to reinforce the mechanical fastening of tissue provided by staples. Such a buttress may prevent the applied staples from pulling through tissue and may otherwise reduce a risk of tissue tearing at or near the site of applied staples. When using a buttress material to reinforce a cut and stapled tissue site, a buttress applicator may be used to load one or more buttresses onto the end effector for subsequent deployment at the cut and stapled tissue site. To preserve the integrity of the buttresses prior to loading and deployment of buttresses at a tissue site, various packaging can be used for the buttresses and/or applicators containing the buttresses. A multi-layered buttress (layers of woven textiles and films, largely incompressible) can be used. A buttress can be applied to the anvil and reload. Additional details on a buttress suitable for use with the present disclosure are described, for example, in U.S. Pat. No. 10,166,023, the disclosure of which is incorporated herein by reference in its entirety.

A buttress can also be applied to an end effector of a surgical stapler. An adhesive layer of the buttress can be used to secure the buttress to the end effector. The buttress can be adhered to the end effector when the end effector is opened. The end effector is then actuated on tissue of a patient, thereby stapling the buttress to the tissue. Additional details on a buttress suitable for use with the present disclosure are described, for example, in U.S. Pat. No. 10,166,023, the disclosure of which is incorporated herein by reference in its entirety.

The adhesive layer of the buttress assemblies can include a pattern to assist in both attachment to the end effector and release from the end effector after cutting and stapling a tissue site. Additional details of a buttress suitable for use with the present disclosure are described, for example, in U.S. Pat. No. 11,033,269, the disclosure of which is incorporated herein by reference in its entirety.

In some embodiments, the staples can be used in combination with a scaffold or a cushion (a tissue thickness compensator), where the staples can be deployed through the scaffold or the cushion into tissue captured against the scaffold or the cushion. In some embodiments, cushions (such as foam cushions) can be sandwiched between layers of films and textiles. Such cushions can be preloaded on the reload only (such that a new cushion is used with a new reload). Suitable scaffolds include knitted elastically deformable, bioabsorbable scaffold described, for example, in U.S. Pat. No. 10,982,360, the disclosure of which is incorporated herein by reference in its entirety.

As discussed above, with some surgical staplers, a surgeon is often required to select the appropriate staples having the appropriate staple height for tissue to be stapled. For example, a surgeon will utilize tall staples for use with thick tissue and short staples for use with thin tissue. In some instances, however, the tissue being stapled does not have a consistent thickness and thus, the staples cannot achieve the desired fired configuration for every section of the stapled tissue (e.g., thick and thin tissue sections). The inconsistent thickness of tissue can lead to undesirable leakage and/or tearing of tissue at the staple site when staples with the same or substantially greater height are used, particularly when the staple site is exposed to intra-pressures at the staple site and/or along the staple line.

Accordingly, various examples of adjuncts are provided that can be configured to compensate for varying thickness of tissue that is captured within fired (deployed) staples to avoid the need to take into account staple height when stapling tissue during surgery. That is, the adjuncts described herein can allow a set of staples with the same or similar heights to be used in stapling tissue of varying thickness (e.g., from thin to thick tissue) while also, in combination with the adjunct, providing adequate tissue compression within and between fired staples. Thus, the adjuncts described herein can maintain suitable compression against thin or thick tissue stapled thereto to thereby minimize leakage and/or tearing of tissue at the staple sites. In addition, exemplary adjuncts described herein may be configured to be essentially fully absorbed in the body over a period of 100 to 300 days depending on implanted location and tissue health.

Alternatively, or in addition, the adjuncts can be configured to promote tissue ingrowth. In various instances, it is desirable to promote the ingrowth of tissue into an implantable adjunct, to promote the healing of the treated tissue (e.g., stapled and/or incised tissue), and/or to accelerate the patient's recovery. More specifically, the ingrowth of tissue into an implantable adjunct may reduce the incidence, extent, and/or duration of inflammation at the surgical site. Tissue ingrowth into and/or around the implantable adjunct may, for example, manage the spread of infections at the surgical site. The ingrowth of blood vessels, especially white blood cells, for example, into and/or around the implantable adjunct may fight infections in and/or around the implantable adjunct and the adjacent tissue. Tissue ingrowth may also encourage the acceptance of foreign matter (e.g., the implantable adjunct and the staples) by the patient's body and may reduce the likelihood of the patient's body rejecting the foreign matter. Rejection of foreign matter may cause infection and/or inflammation at the surgical site.

In general, the adjuncts provided herein are designed and positioned atop a staple cartridge, like staple cartridge 200. When the staples are fired (deployed) from the cartridge, the staples penetrate through the adjunct and into tissue. As the legs of the staple are deformed against the anvil that is positioned opposite the staple cartridge, the deformed legs capture a portion of the adjunct and a portion of the tissue within each staple. That is, when the staples are fired into tissue, at least a portion of the adjunct becomes positioned between the tissue and the fired staple. While the adjuncts described herein can be configured to be attached to a staple cartridge, it is also contemplated herein that the adjuncts can be configured to mate with other instrument components, such as an anvil of a surgical stapler. A person of ordinary skill will appreciate that the adjuncts provided herein can be used with replaceable cartridges or staple reloads that are not cartridge based.

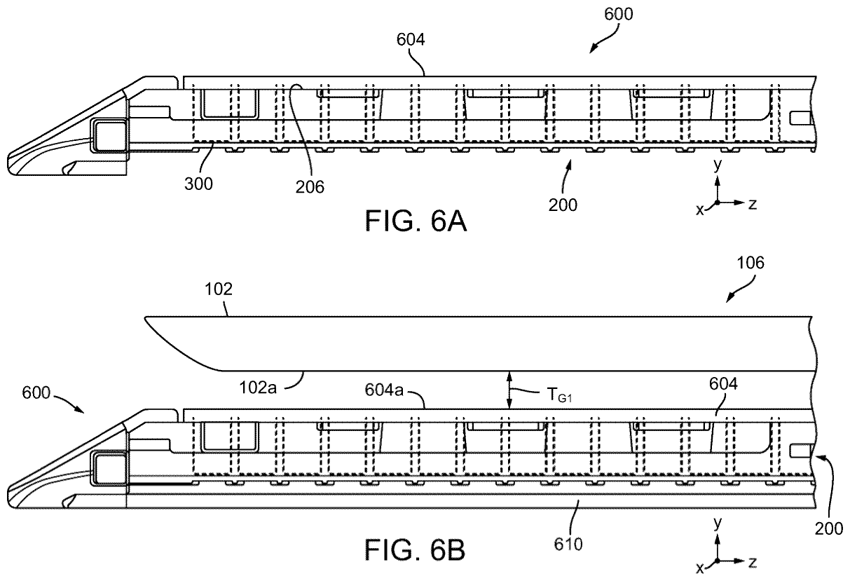

FIGS. 6A-6B illustrate an exemplary example of a stapling assembly 600 that includes a staple cartridge 200 and an adjunct 604. For sake of simplicity, the adjunct 604 is generally illustrated in FIGS. 6A-6B, and various configurations of the adjunct are described in more detail below. As shown, the adjunct 604 is positioned against the staple cartridge 200. While partially obstructed in FIGS. 6A-6B, the staple cartridge 200 includes staples 300 or coated staples 320, that are configured to be deployed into tissue. The staples 300 or coated staples can have any suitable unfirmed (pre-deployed) height.

In the illustrated example, the adjunct 604 can be mated to at least a portion of the top surface or deck surface 206 of the staple cartridge 602. In some examples, the top surface 206 of the staple cartridge 200 can include one or more surface features which can be configured to engage the adjunct 604 to avoid undesirable movements of the adjunct 604 relative to the staple cartridge 200 and/or to prevent premature release of the adjunct 604 from the staple cartridge 200. Exemplary surface features are described further below and in U.S. Patent Publication No. 2016/0106427, which is incorporated by reference herein in its entirety.

FIG. 6B shows the stapling assembly 600 placed within and coupled to the elongate channel 610 of surgical end effector 106. The anvil 102 is pivotally coupled to the elongate channel 610 and is thus moveable between open and closed positions relative to the elongate channel 610, and thus the staple cartridge 200. The anvil 102 is shown in a closed position in FIG. 6B and illustrates a tissue gap Toi created between the staple cartridge 602 and the anvil 612. More specifically, the tissue gap TG1 is defined by the distance between the tissue-compression surface 102a of the anvil 102 (e.g., the tissue-engaging surface between staple forming pockets in the anvil) and the tissue-contacting surface 604a of the adjunct 604. In this illustrated example, both the tissue-compression surface 102a of the anvil 102 and the tissue-contacting surface 604a of the adjunct 604 is planar, or substantially planar (e.g., planar within manufacturing tolerances). As a result, when the anvil 102 is in a closed position, as shown in FIG. 6B, the tissue gap TG1 is generally uniform (e.g., nominally identical within manufacturing tolerances) when no tissue is disposed therein. In other words, the tissue gap TG1 is generally constant (e.g., constant within manufacturing tolerances) across the end effector 106 (e.g., in the y-direction). In other examples, the tissue-compression surface of the anvil can include a stepped surface having longitudinal steps between adjacent longitudinal portions, and thus create a stepped profile (e.g., in the y-direction). In such examples, the tissue gap TG1 can be varied.

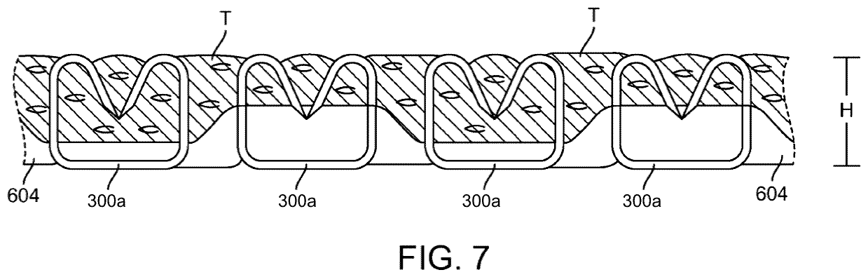

The adjunct 604 is compressible to permit the adjunct to compress to varying heights to thereby compensate for different tissue thicknesses that are captured within a deployed staple. The adjunct 604 has an uncompressed (undeformed), or pre-deployed, height and is configured to deform to one of a plurality of compressed (deformed), or deployed, heights. For example, the adjunct 604 can have an uncompressed height which is greater than the fired height of the staples 300 disposed within the staple cartridge 200 (e.g., the height (H) of the fired staple 300a in FIG. 7). That is, the adjunct 604 can have an undeformed state in which a maximum height of the adjunct 604 is greater than a maximum height of a fired staple (e.g., a staple that is in a formed configuration).

Alternatively, the anvil 102 can have a camber or bend which reduces the tissue gap. This allows the device to grasp tissue when closed more effectively. The E-beam sets the proper tissue gap as it progresses distally. Additional details on an anvil suitable for use with the present disclosure are described, for example, in U.S. Pat. No. 9,586,663, the disclosure of which is incorporated herein by reference in its entirety.

As shown in FIG. 7, when the staples 300 are fired, tissue (T) and a portion of the adjunct 604 are captured by the fired (formed) staples 300a. The fired staples 300a each define the entrapment area therein, as discussed above, for accommodating the captured adjunct 604 and tissue (T). The entrapment area defined by a fired staple 300a is limited, at least in part, by a height (H) of the fired staple 300a.

Staple 300 in its fired (formed) state can have a two-dimensional shape (staple 300a, FIG. 8B) or a three-dimensional shape of staple (staples 300b, FIG. 8A). In staple 300a, the crown (1002a) and both proximal and distal legs (1004a and 1006a) of staple (300a) all lie in a same plane as each other, with the tips of legs laterally aligned with each other. Staple (300a) may be substantially B-shaped in the plane in which crown (1002a) and both legs (1004a and 1006a) reside.

In some embodiments, the staples can be formed into fired (formed) staples having the same height. In some embodiments, the staples can be formed into fired (formed) staples having different height.

On the other hand, the proximal and distal legs (1004b and 1006b) of staple (300b) lie in different planes from each other and from crown (1002b), with the tips of legs laterally offset from each other and from crown (1002b) on opposed sides of crown (1002b). More particularly, proximal leg (1004b) skews laterally outwardly away from the longitudinal axis relative to crown (1002b), and distal leg (1006b) skews laterally inwardly toward the longitudinal axis relative to crown (1002b). Suitable staples are described, for example, in U.S. Pat. Publ. No. 2024/0341761, the disclosure of which is incorporated herein by reference in its entirety.

A coated implant, such as staple, may be coated to delay its degradation in vivo and/or reduce friction between the implant body and an external object (e.g., tissue of the patient). Coated staple may include an implant body 300 (e.g., a staple or staple body) and a coating. The implant body may include or be formed of an absorbable metal such as calcium, magnesium, or zinc or alloy thereof.

In some embodiments, the staple comprises an absorbable metal that is significantly, principally, or substantially composed of zinc.

In some embodiments, the staple comprises an absorbable metal that is significantly, principally, or substantially composed of magnesium.

The coating may include a copolymer comprising a residue that is a succinate-based monomer.

The succinate-based polymer coating may be disposed on at least a portion of a surface of the implant body 300 such that the coating delays degradation of the at least one portion of the implant body 300 in vivo.

The succinate-based polymer has water barrier properties due to cis: trans alkene rotation in the backbone. Ester linkages can be tailored to control the degradation. This type of polymers is a good candidate for Mg alloy applications including staples and 3D printed parts for implantable applications.

The succinate-based polymers include ones described in U.S. Pat. No. 10,968,303 to Becker et al. and U.S. Patent Application Publication No. 2024/0018302 to Becker et al., which are incorporated herein by reference in their entireties.

Advantageously, the coating comprising succinate-based copolymers can be applied to the Mg alloy to address the limitations described in the Background section. As a result of coating, the sensitivity of the magnesium alloy to physiological conditions can be minimized; the induction period for magnesium alloy implant absorption can be tailored; the spatial resolution on the absorption profile of the magnesium alloy in vivo can be achieved; the active pharmaceutical ingredients (APIs) can be included in the coating; and the feature size can be reduced for longer-term implants.

Provided herein is a coated staple comprising:
a staple 300; and
a coating comprising a copolymer comprising a residue that is a succinate-based monomer.

In some embodiments, the staple comprises an absorbable metal that is significantly, principally, or substantially composed of zinc.

In some embodiments, the staple comprises an absorbable metal that is significantly, principally, or substantially composed of magnesium.

Provided herein is a coated staple comprising:
a staple 300 comprised substantially of magnesium; and
a coating comprising a copolymer comprising a residue that is a succinate-based monomer.

Further provided herein is a coated staple comprising:
a staple 300; and
a coating comprising a copolymer comprising a residue that is a succinate-based monomer, wherein the coating is disposed on at least a portion of a surface of the staple, and the coating has an elongation at break (¿break) of at least about 11%.

Further provided herein is a coated staple comprising:
a staple 300 comprised substantially of magnesium; and
a coating comprising a copolymer comprising a residue that is a succinate-based monomer, wherein the coating is disposed on at least a portion of a surface of the staple, and the coating has an elongation at break (break) of at least about 11%.

In some embodiments, the succinate-based coating has an elongation at break (ε_{break}) of at least about 11%, at least about 20%, at least about 50%, at least about 100%, about 11% to about 500%, about 11% to about 400%, about 11% to about 300%, about 11% to about 200%, or about 11% to about 150%.

In some embodiments, the succinate-based polymer coating has a Young's modulus (E) of about 40 MPa to about 110 MPa. In some embodiments, the succinate-based polymer coating has a Young's modulus (E) of about 5 MPa to about 200 MPa, about 10 MPa to about 200 MPa, about 20 MPa to about 200 MPa, about 30 MPa to about 200 MPa, about 40 MPa to about 200 MPa, about 5 MPa to about 150 MPa, about 10 MPa to about 150 MPa, about 20 MPa to about 150 MPa, about 30 MPa to about 150 MPa, about 40 MPa to about 150 MPa, about 5 MPa to about 110 MPa, about 10 MPa to about 110 MPa, about 20 MPa to about 110 MPa, about 30 MPa to about 110 MPa, about 40 MPa to about 110 MPa, about 5 MPa to about 80 MPa, about 10 MPa to about 80 MPa, about 20 MPa to about 80 MPa, about 30 MPa to about 80 MPa, about 40 MPa to about 80 MPa, about 5 MPa to about 60 MPa, about 10 MPa to about 60 MPa, about 20 MPa to about 60 MPa, about 30 MPa to about 60 MPa, or about 40 MPa to about 60 MPa.

In some embodiments, the succinate-based polymer coating has an ultimate tensile strength (UTS) of about 20 MPa to about 60 MPa, about 20 MPa to about 80 MPa, about 20 MPa to about 100 MPa, about 20 MPa to about 120 MPa, about 30 MPa to about 60 MPa, about 30 MPa to about 80 MPa, about 30 MPa to about 100 MPa, about 30 MPa to about 120 MPa, about 40 MPa to about 60 MPa, about 40 MPa to about 80 MPa, about 40 MPa to about 100 MPa, or about 40 MPa to about 120 MPa.

In some embodiments, the succinate-based polymer has about 1% to about 40% CSS. In some embodiments, the succinate-based polymer has about 5% to about 40% CSS. In some embodiments, the succinate-based polymer has about 10% to about 40% CSS. In some embodiments, the succinate-based polymer has about 15% to about 40% CSS. In some embodiments, the succinate-based polymer has about 5%, about 10%, about 15%, about 20%, about 25%, about 30%, about 35%, or about 40% CSS.

Also provided herein is a coated staple comprising:
a staple 300; and
a coating comprising a copolymer comprising a residue that is a succinate-based monomer, wherein the coating is disposed on at least a portion of a surface of the staple, and the coating has a glass transition temperature (Tg) of about −20° C. to about 10° C.

Also provided herein is a coated staple comprising:
a staple 300 comprised substantially of magnesium; and
a coating comprising a copolymer comprising a residue that is a succinate-based monomer, wherein the coating is disposed on at least a portion of a surface of the staple, and the coating has a glass transition temperature (Tg) of about −20° C. to about 10° C.

In some embodiments, the succinate-based polymer coating has a glass transition temperature (Tg) of about −30° C. to about 10° C., about −20° C. to about 10° C., about −15° C. to about 10° C., about −10° C. to about 10° C., 30° C. to about 5° C., about −20° C. to about 5° C., about −15° C. to about 5° C., about −10° C. to about 5° C., 30° C. to about 0° C., about −20° C. to about 0° C., about −15° C. to about 0° C., about −10° C. to about 0° C.

In some embodiments, the succinate-based polymer coating has a glass transition temperature (Tg) of about −8° C.

Provided herein is a coated staple comprising:
a staple 300; and
a coating comprising a copolymer comprising a residue that is a succinate-based monomer, wherein the coating is disposed on at least a portion of a surface of the staple, wherein when the coating has a thickness of about 2 μm to about 22 μm, the water uptake of the coating is less than about 3.3% by weight.

Provided herein is a coated staple comprising:
a staple 300 comprised substantially of magnesium; and
a coating comprising a copolymer comprising a residue that is a succinate-based monomer, wherein the coating is disposed on at least a portion of a surface of the staple, wherein when the coating has a thickness of about 2 μm to about 22 μm, the water uptake of the coating is less than about 3.3% by weight.

In some embodiments, when the coating has a thickness of about 2 μm to about 22 μm, the water uptake of the coating is less than about 0.5%, less than about 1%, less than about 1.5%, less than about 1.9%, less than about 2%, less than about 2.5%, less than about 3%, or less than about 3.3% by weight.

In some embodiments, when the coating has a thickness of about 6 μm, the water uptake of the coating is about 0.2% by weight.

In some embodiments, when the coating has a thickness of about 2 μm to about 10 μm, the water uptake of the coating is less than about 1.9%. In some embodiments, when the coating has a thickness of about 10 μm to about 22 μm, the water uptake of the coating is about 1.9% to about 3.3% by weight.

The coating may have elongation that is greater than elongation of the staple.

The ability to tailor the staple absorption may be controlled via coating thickness. As used herein, the term “coating thickness” or “thickness of the coated staple” refers to the overall thickness of the coated staple (thickness of staple with coating). The coatings present on the coated staple can have a uniform thickness or the thickness can very throughout the coating. When the coating has ununiform thickness, such coating can be defined by an average (targeted) thickness and a minimum thickness.

The coating may have a minimum thickness of from about 1 micron to about 11 microns, from about 1 micron to about 10 microns, from about 1 micron to about 9 microns, from about 1 micron to about 8 microns; from about 1 micron to about 7 microns; from about 1 micron to about 6 microns; from about 1 micron to about 5 microns, from about 1 micron to about 4 microns, from about 1 micron to about 3 microns, from about 1 micron to about 2 microns, from about 2 microns to about 10 microns, from about 3 microns to about 9 microns, from about 4 microns to about 8 microns, from about 5 microns to about 7 microns, from about 2 microns to about 3 microns, from about 3 microns to about 4 microns, from about 4 microns to about 5 microns, from about 5 microns to about 6 microns, from about 6 microns to about 7 microns, from about 7 microns to about 8 microns, from about 8 microns to about 9 microns, or from about 9 microns to about 10 microns. The coating may have a minimum thickness of at least about 1 micron, at least about 2 microns, at least about 3 microns, at least about 4 microns, at least about 5 microns, at least about 6 microns, at least about 7 microns, at least about 8 microns, at least about 9 microns, at least about 10 microns, or at least about 11 microns.

The coating may have an average (targeted) thickness of from about 5 microns to about 22 microns, from about 6 microns to about 22 microns, from about 7 microns to about 21 microns, from about 8 microns to about 20 microns; from about 9 microns to about 19 microns; from about 10 microns to about 18 microns; from about 11 microns to about 17 microns, from about 12 microns to about 16 microns, from about 13 microns to about 15 microns, from about 6 microns to about 21 microns, from about 6 microns to about 20 microns, from about 6 microns to about 19 microns, from about 6 microns to about 18 microns, from about 6 microns to about 17 microns, from about 6 microns to about 16 microns, from about 6 microns to about 15 microns, from about 6 microns to about 14 microns, from about 6 microns to about 13 microns, from about 6 microns to about 12 microns, from about 6 microns to about 11 microns, from about 6 microns to about 10 microns, from about 6 microns to about 9 microns, from about 7 microns to about 19 microns, from about 8 microns to about 19 microns, from about 9 microns to about 19 microns, from about 10 microns to about 19 microns, from about 11 microns to about 19 microns, from about 12 microns to about 19 microns, from about 13 microns to about 19 microns, from about 14 microns to about 19 microns, or from about 15 microns to about 19 microns. The coating may have an average (targeted) thickness CH of at least about 5 microns, at least about 6 microns, at least about 7 microns, at least about 8 microns, at least about 9 microns, at least about 10 microns, at least about 11 microns, at least about 12 microns, at least about 13 microns, at least about 14 microns, at least about 15 microns, at least about 16 microns, at least about 17 microns, at least about 18 microns, or at least about 19 microns.

The implant body 300 may comprise a magnesium alloy.

The implant body 300 may be selected from the group consisting of a surgical staple, a bone screw, a trauma plate, a craniomaxillofacial (CMF) implant, a bioprinting scaffold, and a drug delivery or load-bearing absorbable implant.

The implant body 300 may be a surgical staple.

The implant body 300 may be absorbable, and/or the coating is absorbable.

In some embodiments, at least one drug is embedded in the coating.

The coating 312 may be for use in reducing sensitivity of the implant body to physiological conditions or tailoring an induction period of at least a portion of the implant body.

The coating 312 may have a coating degradation profile that is insensitive to pH.

In some embodiments, the coating may have a coating degradation profile that is substantially insensitive to physiological or tissue pH.

The coated implant or coated portion of the implant may degrade slower than the implant body 300 or the uncoated portion of the implant body 300.

The succinate-based polymer may comprise about 10 mole % to about 40 mole % of the residue that is a succinate-based monomer.

The succinate-based monomer may have the formula of:

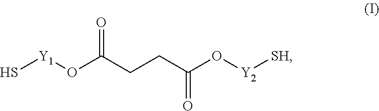

wherein Y₁ and Y₂ are each independently alkylene, ethoxy, arylene, or any combination thereof.

The succinate-based polymer may comprise a residue of C₁ to C₁₂ bis-propriolate.

The succinate-based polymer may comprise a residue of C₁ to C₁₂ bis-propriolate having the formula of:

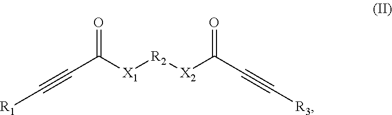

where R₁ and R₃ are each independently H, alkyl, or aryl group; R₂ is a covalent bond, alkylene, ethoxy, arylene, or any combination thereof; and X₁ and X₂ are each independently O, NH, or S.

An example of the synthesis of a succinate-based polymer is shown below:

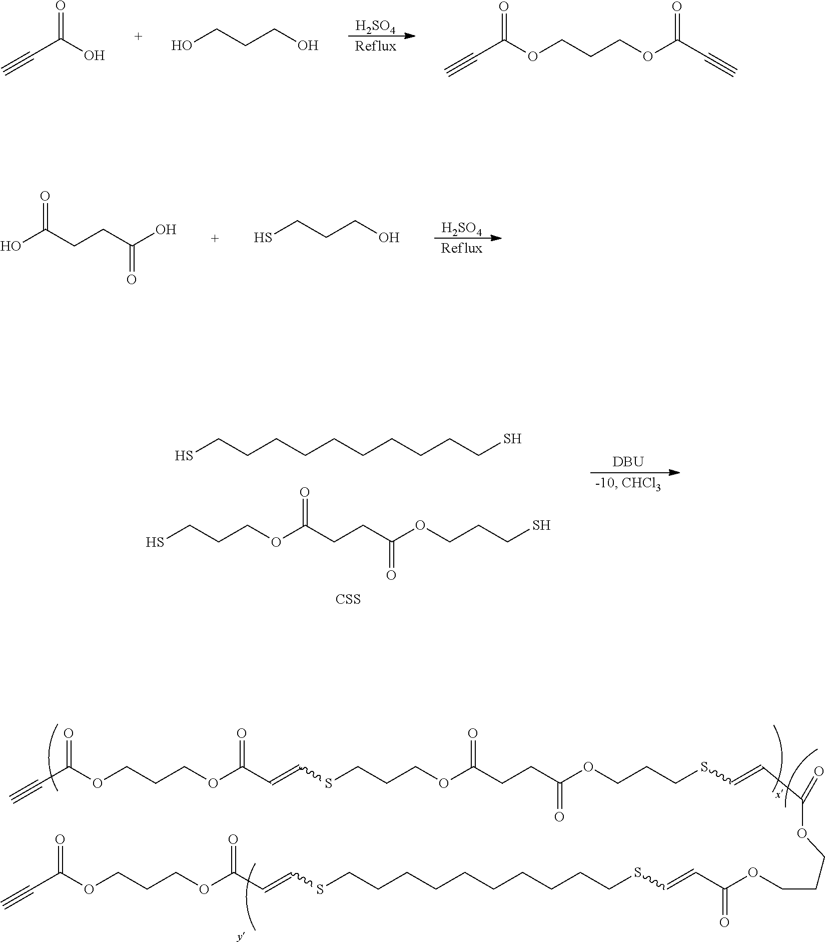

The preparation method was disclosed in McDonald, S. M., Yang, Q., Hsu, Y H. et al. Resorbable barrier polymers for flexible bioelectronics. Nat. Commun. 14, 7299 (2023). https://doi.org/10.1038/s41467-023-42775-5, which is incorporated by reference.

The succinate-based polymer may comprise a residue of a multi-functional thiol having a formula of:

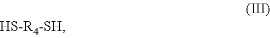

wherein R₄ is alkylene, ethoxy, arylene, or any combination thereof.

The succinate-based polymer may have a formula of:

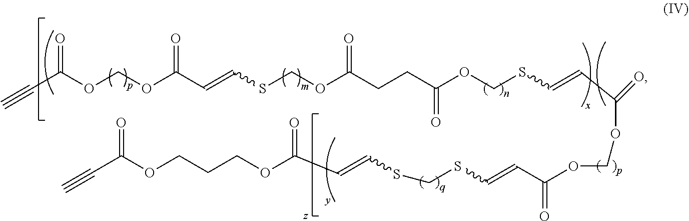

wherein each of m, n, p, and q is independently an integral of 1-20, each of x and y is independently a mole fraction between 0 and 1, the total of x and y is 1, and z is an integer from 5 to 5000. In some embodiments, x may be 0.05 to 0.4, 0.05 to 0.35, 0.05 to 0.3, 0.05 to 0.25, 0.1 to 0.4, or 0.15 to 0.4. In some embodiments, each of m, n, p, and q is independently an integral of 1-12. In some embodiments, each of m, n, p, and q is independently an integral of 1-10. In some embodiments, z is an integer from 5 to 3000, 5 to 2000, 5 to 500, 5 to 300, 5 to 200, 5 to 100, 5 to 50, 10 to 5000, 10 to 3000, 10 to 2000, 10 to 1000, 10 to 500, 10 to 300, 10 to 200, 10 to 100, or 10 to 50.

The succinate-based polymer may have a formula of:

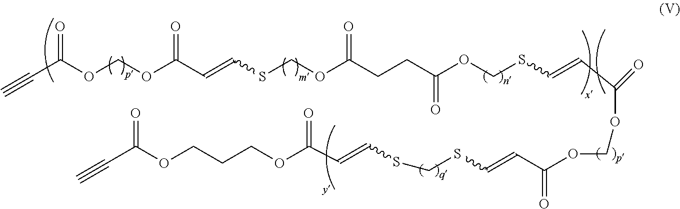

wherein each of m′, n′, p′, and q′ is independently an integral of 1-20, each of x′ and y′ is independently an integer from 5 to 5000. In some embodiments, each of m′, n′, p′, and q′ is independently an integral of 1-12. In some embodiments, each of m′, n′, p′, and q′ is independently an integral of 1-10. In some embodiments, z′ is an integer from 5 to 3000, 5 to 2000, 5 to 500, 5 to 300, 5 to 200, 5 to 100, 5 to 50, 10 to 5000, 10 to 3000, 10 to 2000, 10 to 1000, 10 to 500, 10 to 300, 10 to 200, 10 to 100, or 10 to 50.

The succinate-based polymer may have a formula of:

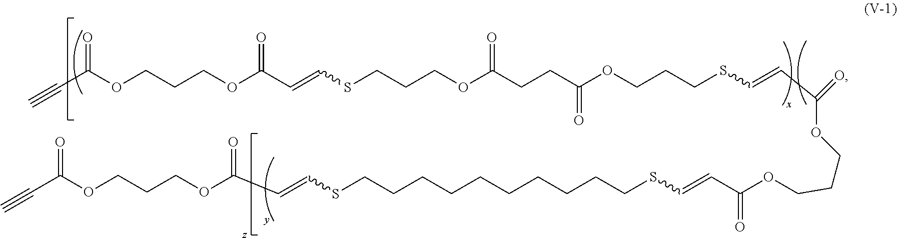

wherein x, y, and z are defined above.

The succinate-based polymer may have a formula of:

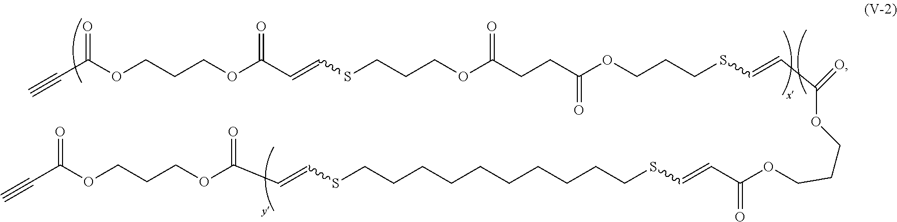

wherein x, y, and z are defined above.

The succinate-based polymer described in Example 1 below (CSS polymer; 25% CSS), has the formula of:

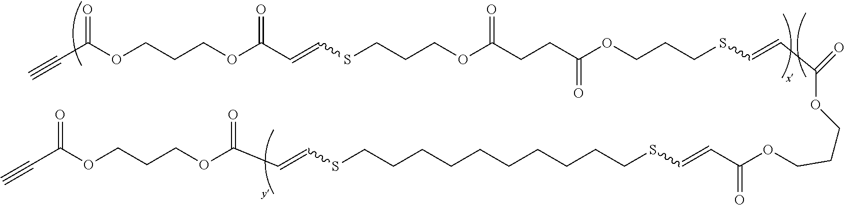

wherein x′ is 0.25.

The coated staple may comprise magnesium or magnesium alloy.

The coated staple may comprise substantially of magnesium.

The coated staple may further comprise one or more metallic elements selected from an alkali metal, an alkaline earth metal, a transition metal, or lanthanides, wherein the one or more metallic elements selected from aluminum, beryllium, calcium, cerium, copper, gadolinium, lithium, iron, manganese, molybdenum, neodymium, nickel, silver, strontium, thorium, tin, titanium, vanadium, yttrium, zirconium, dysprosium, or zinc.

A staple can be characterized by a radius of curvature. In FIG. 3 a staple comprises a crown 1002, a first leg 1004, and a second leg 1006. A radius of curvature 1008 is between the crown 1002 and each of the first leg 1004 and the second leg 1006.

The radius between the crown and the staple legs is an important design choice for a staple. It influences staple strength due to the amount of work hardening that happens in the region. This strength can help support the staple legs as they pass through the tissue, during folding of the staple legs, and after formation while residing in situ.

In some embodiments, the radius of curvature 1008 is greater than 0.005 inches, greater than 0.006 inches, greater than 0.007 inches, greater than 0.008 inches, greater than 0.009 inches, or greater than 0.01 inches. In other embodiments, the radius of curvature 1008 is equal to 0.004 inches, 0.005 inches, 0.006 inches, 0.007 inches, 0.008 inches, 0.009 inches, or 0.01 inches.

A method of making a coated staple is also provided herein. The method includes coating at least a portion of a surface of a staple 300 with the copolymer comprising a residue that is a succinate monomer.

The coating the at least a portion of the surface of the staple can be conducted via inkjet printing, direct depositing, thermal spraying, cold dynamic spraying, cold spraying, electrospraying, ultrasonic spray coating, dip coating, chemical vapor deposition, screen printing, spin coating, reel-to-reel coating, or combinations thereof.

A surgical device 100 is also provided herein. The surgical device includes a cartridge body 200. The cartridge body includes a deck; and a staple cavity defined in the deck. The staple cavity can include any of the coated staples described above.

The surgical device 100 can further include an end effector 106. The end effector includes a first jaw; and a second jaw, wherein at least one of said first jaw and said second jaw is movable between an open position and a clamped position. The cartridge body 200 is positionable within the second jaw.

In some embodiments, the surgical devise is a handheld stapler, robotics stapler, laparoscopic stapler, or open stapler.

As will be appreciated by one skilled in the art, the embodiments described above are cited by way of example, and the present invention is not limited by what has been particularly shown and described hereinabove. Rather, the scope of the invention includes both combinations and sub combinations of the various features described hereinabove, as well as variations and modifications thereof which would occur to persons skilled in the art upon reading the foregoing description and which are not disclosed in the prior art.

In some examples, disclosed devices (e.g., end effector, surgical adjunct, and/or staple cartridges) and methods involving one or more disclosed devices may involve one or more of the following clauses:

Clause 1. A coated staple comprising:
a staple 300; and
a coating comprising a copolymer comprising a residue that is a succinate-based monomer,
wherein the coating is disposed on at least a portion of a surface of the staple, and the coating has an elongation at break (ε_{break}) of at least about 11%.

Clause 2. A coated staple comprising:
a staple 300 comprised substantially of magnesium; and
a coating comprising a copolymer comprising a residue that is a succinate-based monomer,
wherein the coating is disposed on at least a portion of a surface of the staple, and the coating has an elongation at break (ε_{break}) of at least about 11%.

Clause 3. The coated staple of any one of clauses 1-2, wherein the coating has a Young's
modulus (E) of about 40 MPa to about 110 MPa, and/or wherein the coating has an ultimate
tensile strength (UTS) of about 20 MPa to about 60 MPa.

Clause 4. A coated staple comprising:
a staple 300; and
a coating comprising a copolymer comprising a residue that is a succinate-based monomer,
wherein the coating is disposed on at least a portion of a surface of the staple, and the coating has an elongation at break (ε_{break}) of at least about 11%.

Clause 5. A coated staple comprising:
a staple 300 comprised substantially of magnesium; and
a coating comprising a copolymer comprising a residue that is a succinate-based monomer,
wherein the coating is disposed on at least a portion of a surface of the staple, and the coating has a glass transition temperature (Tg) of about −20° C. to about 10° C.

Clause 6. The coated staple of any one of clauses 1-5, wherein the coating has a glass transition temperature (Tg) of about −8° C.

Clause 7. A coated staple comprising:
a staple 300; and
a coating comprising a copolymer comprising a residue that is a succinate-based monomer, wherein the coating is disposed on at least a portion of a surface of the staple, wherein when the coating has a thickness of about 2 μm to about 22 μm, the water uptake of the coating is less than about 3.3% by weight.

Clause 8. A coated staple comprising:
a staple 300 comprised substantially of magnesium; and
a coating comprising a copolymer comprising a residue that is a succinate-based monomer, wherein the coating is disposed on at least a portion of a surface of the staple, wherein when the coating has a thickness of about 2 μm to about 22 μm, the water uptake of the coating is less than about 3.3% by weight.

Clause 9. The coated staple of any one of clauses 1-8, wherein the coating has a thickness of about 6 μm and/or wherein the coating wherein the water uptake of the coating is less than 1.0% by weight.

Clause 10. The coated staple of any one of clauses 1-9, wherein: the coating is configured to delay degradation of the at least one portion of the staple 300 in vivo; the staple 300 is absorbable; and/or the coating is absorbable.

Clause 11. The coated staple of any of clauses 1-10, wherein the copolymer comprises about 10 mole % to about 40 mole % of the residue that is a succinate-based monomer.

Clause 12. The coated staple of any of clauses 1-11, wherein the residue that is a succinate-based monomer is a product of a succinic acid and a hydroxyl thiol.

Clause 13. The coated staple of any of clauses 1-12, wherein the succinate-based monomer has the formula of:

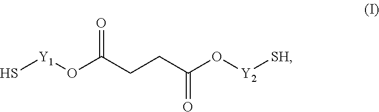

wherein Y₁ and Y₂ are each independently alkylene, ethoxy, arylene, or any combination thereof.

Clause 14. The coated staple of any one of clauses 1-13, wherein the copolymer further comprises a residue of C₁ to C₁₂ bis-propriolate.

Clause 15. The coated staple of any one of clauses 1-14, wherein the copolymer further comprises a residue of C₁ to C₁₂ bis-propriolate having the formula of:

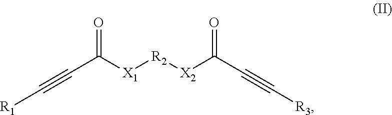

where R₁ and R₃ are each independently H, alkyl, or aryl group; R₂ is a covalent bond, alkylene, ethoxy, arylene, or any combination thereof, and X₁ and X₂ are each independently O, NH, or S.

Clause 16. The coated staple of any one of clauses 1-15, wherein the copolymer further comprises a residue of a multi-functional thiol having a formula of HS—R₄—SH (III), wherein R₄ is alkylene, ethoxy, arylene, or any combination thereof

Clause 17. The coated staple of any one of clauses 1-16, wherein the copolymer has a formula of:

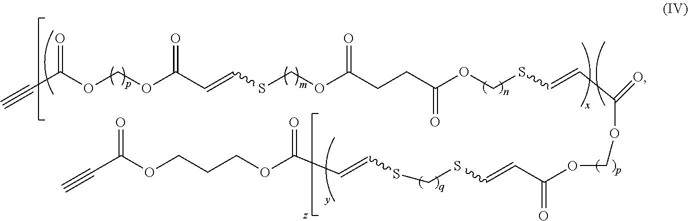

wherein each of m, n, p, and q is independently an integral of 1-12, each of x and y is independently a mole fraction between 0 and 1, the total of x and y is 1, and z is an integer from 10 to 500,
or

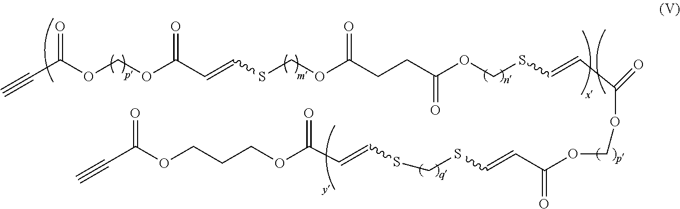

wherein each of m′, n′, p′, and q′ is independently an integral of 1-12, each of x′ and y′ is independently an integer from 10 to 500.

Clause 18. The coated staple of any one of clauses 1-17, wherein the copolymer has the formula of:

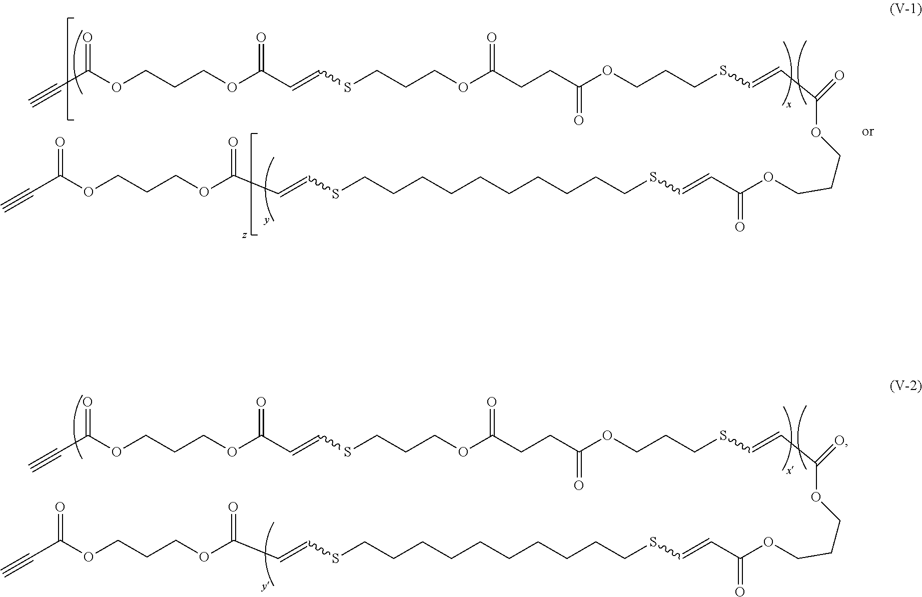

wherein x and y are mole fractions and the total of x and y is 1, z is an integer from 10 to 500, x′ and y′ are each independently an integer from 10 to 500, optionally wherein x is a mole fraction from about 0.05 to about 0.40.

Clause 19. The coated staple of any one of clauses 1, 3, 4, 6, 7, and 9-18, wherein the staple comprises an absorbable metal that is significantly, principally, or substantially composed of magnesium.

Clause 20. The coated staple of any one of clauses 1, 3, 4, 6, 7, and 9-18, wherein the staple comprises an absorbable metal that is significantly, principally, or substantially composed of zinc.

Clause 21. The coated staple of any one of clauses 1-19, further comprising one or more metallic elements selected from an alkali metal, an alkaline earth metal, a transition metal, or lanthanides, wherein the one or more metallic elements selected from aluminum, beryllium, calcium, cerium, copper, gadolinium, lithium, iron, manganese, molybdenum, neodymium, nickel, silver, strontium, thorium, tin, titanium, vanadium, yttrium, zirconium, dysprosium, or zinc.

Clause 22. The coated staple of any one of clauses 1-18 and 20, further comprising one or more metallic elements selected from an alkali metal, an alkaline earth metal, a transition metal, or lanthanides, wherein the one or more metallic elements selected from aluminum, beryllium, calcium, cerium, copper, gadolinium, lithium, iron, manganese, molybdenum, neodymium, nickel, silver, strontium, thorium, tin, titanium, vanadium, yttrium, zirconium, dysprosium, or magnesium.

Clause 23. The coated staple of any one of clauses 1-22, comprising a crown 1002, a first leg 1004, and a second leg 1006, a radius of curvature 1008 between the crown 1002 and each of the first leg 1004 and the second leg 1006 being greater than or equal to 0.005 inches.

Clause 24. A method of making the coated staple of any one of clauses 1-23, comprising:
coating at least a portion of a surface of the staple with the copolymer comprising a residue that is a succinate monomer, wherein coating is conducted via electrospinning, electrospray, dip coating, thermal spray, screen printing, an extrusion process comprising pultrusion, direct deposition, laser sintering, overmolding, spray coating, chemical vapor deposition, reel-to-reel coating, or combinations thereof.

Clause 25. A surgical device 100, comprising:
a cartridge body 200, comprising:
   a deck; and
   a staple cavity defined in the deck, the staple cavity comprising the coated staple of any
   one of clauses 1-23.

Clause 26. The surgical device 100 of clause 25 further comprising:
an end effector 106 comprising:
   a first jaw; and
   a second jaw, wherein at least one of said first jaw and said second jaw is movable between an open position and a clamped position, and wherein the cartridge body 200 is positionable within the second jaw.

### EXAMPLES

The following Examples are presented to illustrate various aspects of the present disclosure, but are by no means intended to limit its scope.

### Example 1. Coated Staples

In a staple, the radius of curvature at 0.005 inches resulted in fractures in the staple in the curvature region during formation. The radius of curvature at 0.01 inches did not have the same facture problem. The staples used in the examples have the radius of curvature at 0.02 inches.

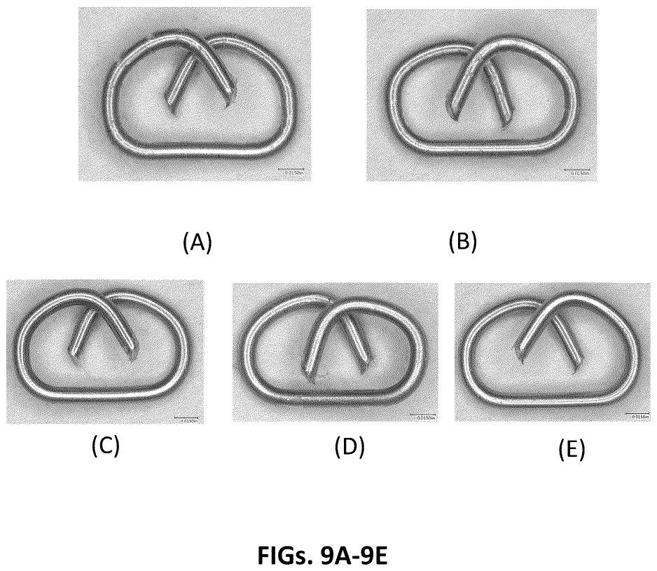

Staples were made of a magnesium alloy, further comprising other metals, such as lithium, zinc, calcium, manganese, etc. For example, the staples are described in European Publication No. 3975942B1, the disclosure of which is incorporated herein by reference in its entirety. Staples were coated with about 6 μm layer of low molecular weight poly(L-lactic acid) (LMW PLLA) (Resomer L 206S from Aldrich), high molecular weight poly(L-lactic acid) (HMW PLLA) (Resomer L 210S from Aldrich), a succinate-based polymer (CSS polymer; 25% CSS), polycaprolactone (PCL) (Resomer C 212 from Evonik), and polyester polyurethane (PEU) (Cyprex PEU V8 from Viamer) and characterized (Table 1 and FIGS. 9A-9E). On the staples coated with LMW PLLA and HMW PLLA, wrinkles and coating tear off were observed (FIGS. 9A-9B). Tip extensions were observed on the staples coated with CSS polymer and PCL (FIGS. 9C-9D). Overall, staples coated with CSS polymer, PCL, and PEU have shown good appearance (FIGS. 9C-9E), with the ε_{break} of a coated wire at a higher level than the ε_{break} of a non-coated wire (about 13%) and relatively low Young's Modulus (indicating less stiffness). The CSS polymer used in the examples is represented by Formula (V-2). The CSS polymer with 15% CSS was also tested and showed good appearance.

PEU1 and PEU 2 are amino acid-based poly(ester urea) having the following structure:

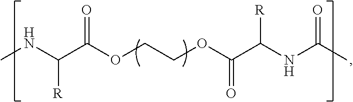

where R is amino acid side chain (Dziewior et al., “Poly(ester Urea) s: Synthesis, Material Properties, and Biomedical Applications,” Progress in Polymer Science 156:101866 (2024), which is incorporated herein by reference in its entirety).

CSS is a degradable dithiol derivative of succinic acid having the structure:

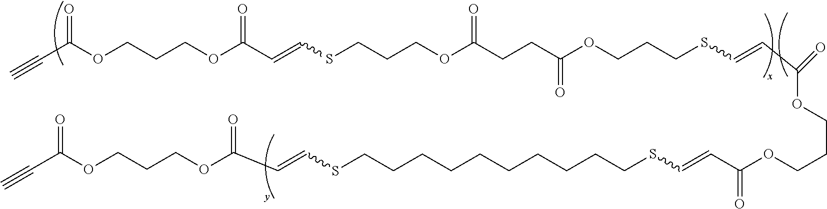

(McDonald et al., “Resorbable Barrier Polymers for Flexible Bioelectronics,” Nature Communications 14:7299 (2023), which is incorporated herein by reference in its entirety).

TABLE 1
Properties of Coated Staples

| | | | Ultimate |
|---|---|---|---|
| | Elongation | Young's | Tensile |
| | At Break | Modulus (E), | Strength |
| Coating | (ε_{break}), % | Mpa | (UTS), MPa |
| LMW PLLA | <10 | 3000-3500 | 55-65 |
| HMW PLLA | 10 | 3700-4700 | 75-85 |
| CSS polymer | 1506 ± 37 | 67.2 ± 1.7 | 25 ± |
| (25% CSS) | | | 3.4 to 54 |
| PCL | 180 | 500-600 | 15-25 |
| PEU | 184.17 ± 22.05 | 1174.61 ± 363.59 | 78.5 ± 11.5 |

### Example 2. Staple Aging Test 1

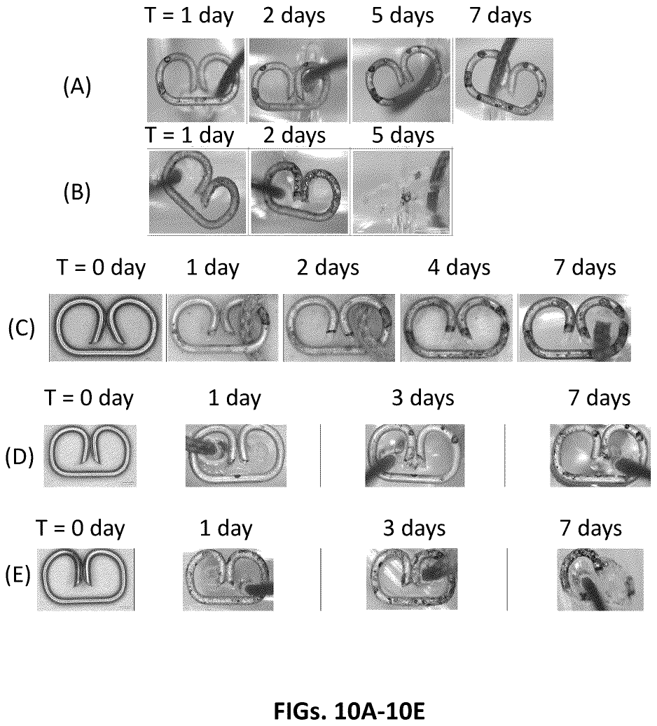

Staple aging tests were performed by submerging the coated staples (coating thickness of about 6 μm) in Simulated Gastric Fluid (SGF) at 22° C. (FIG. 10A) and 38° C. (FIGS. 10B-10E) and observing staple degradation over 7 days. No wire was observed after soaking a staple coated with PEU1 in SFG at 38° C. after 5 days (FIG. 10B). Staples covered with CSS Polymer (25% CSS) (FIG. 10C) and PCL coating (FIG. 10D) retained the shape at body temperature after 7 days. The glass transition temperatures (T_{g}) for different coatings were measured with results shown in Table 2.

TABLE 2
Glass Transition Temperature (Tg)

| Coating | Tg, ° C. |
|---|---|
| PEU1 | 30.1 |
| CSS Polymer | −8 to −9 |
| (25% CSS) | |
| PCL | −60 |
| PEU2 | 49.9 |

### Example 3. Staple Aging Test 2

Additional staple aging tests were performed by submerging the coated staples (coating thickness of about 6 μm) in SGF at about 37° C. The CSS polymer has a minimum thickness of coating at about 2 μm. The staple aging performance was evaluated by the percentage of intactstaple legs (% passing staple legs) after day 7, day 14 and day 28 of aging in Table 3. The water uptake (WU) of the coating was measured (Table 3). The higher the percentage of passing staple legs, the better the coating performance to protect the Mg alloy staples. The polymer coating of lower water update has better staple aging performance. Among the coating of the same thickness, the CSS polymer shows the best polymer coating performance in SGF, followed by PCL, PLLA, and PEU2 in a decreasing order polymer coating performance.

TABLE 3
Water Uptake (WU) and Percentage of Passing Staple Legs
for Coated Staples (Coating Thickness of About 6 μm)

| | | % passing | % passing | % passing |
|---|---|---|---|---|
| | | staple legs | staple legs | staple legs |
| | Water | after aging | after aging | after aging |
| Polymer | uptake | 7 days | 14 days | 28 days |
| coating | (WU) | in SGF | in SGF | in SGF |
| Parylene | <0.1% | 67 | 33 | 0 |
| PLLA | 0.8-1.2% | 42 | 0 | 0 |
| PEU2 | 1.9-3.3% | 0 | 0 | 0 |
| PCL | 0.75% | 67 | 50 | 27 |
| CSS Polymer | 0.2% | 100 | 92 | 92 |
| (25% CSS) | | | | |

### Example 4. Staple Aging Test 3

Further staple aging tests were performed by submerging the coated staples (coating thickness of about 6 μm or about 19 μm) in SGF at about 37° C. The staple aging performance was evaluated by the percentage of passing legs after day 7, day 14 and day 28 of aging in Table 4. By increasing the coating thickness, the percentage of passing staple legs significantly improved. Coating of water uptake greater than 1.9% had a high percentage of passing staple legs when the coating was thick.

TABLE 4
Water Uptake (WU) and Percentage of Passing Staple Legs for Coated
Staples (Coating Thickness of About 6 μm or about 19 μm)

| | | | | | % passing | % passing | % passing |
|---|---|---|---|---|---|---|---|
| | | | | | staple legs | staple legs | staple legs |
| | Water | | | | after aging | after aging | after aging |
| Polymer | uptake | Coating | Minimum | | 7 days in | 14 days in | 28 days in |
| coating | (WU) | thickness | thickness | | SGF | SGF | SGF |
| PEU2 | 1.9-3.3% | 6 μm | 2.5-3 | μm | 0 | 0 | 0 |
| PEU2 | 1.9-3.3% | 19 μm | 10 | μm | 100 | 67 | 0 |
| PCL | 0.75% | 6 μm | 2.5-3 | μm | 67 | 50 | 27 |
| PCL | 0.75% | 19 μm | 10 | μm | 92 | 92 | 83 |

### Example 5. Staple Aging Test 4

Staple aging tests were performed by submerging the CSS polymer coated staples (coating thickness of about 6 μm, about 12 μm or about 19 μm) in SGF at about 37° C. Different trials used different batches of staple wires. The staple aging performance was evaluated by the percentage of passing legs after day 7, day 14 and day 28 of aging in Table 5. The increased thickness of CSS polymer improved coating performance by prolonging protection. The coating thickness of CSS polymer did not impact the coating performance as pronounced as to PEU or PCL. The best-performing CSS polymer has the thickest coating at 19 μm thickness. Thinner coating at 6 μm or 12 μm thickness also showed good staple aging results.

TABLE 5
Percentage of Passing Staple Legs for CSS Polymer
Coated Staples (Coating Thickness of About 6
μm, About 12 μm, or about19 μm)

| | | | | % passing | % passing | % passing |
|---|---|---|---|---|---|---|
| | | | | staple legs | staple legs | staple legs |
| | | | | after aging | after aging | after aging |
| Coating | Minimum | | Coating | 7 days | 14 days | 28 days |
| thickness | thickness | | Trial | in SGF | in SGF | in SGF |
| 6 μm | 2.5-3 | μm | Trial 1 | 100 | 92 | 50 |
| | | | Trial 2 | 100 | 92 | 92 |
| | | | Trial 3 | 67 | 67 | 67 |
| 12 μm | 5.6 | μm | Trial 1 | 83 | 83 | 83 |
| | 8.3 | μm | Trial 2 | 100 | 100 | 100 |
| 19 μm | 8.5 | μm | Trial 4 | 100 | 100 | 100 |
| | 11.7 | μm | Trial 5 | 100 | 83 | 83 |

### Example 6. Electron Beam (Ebeam) Sterilization Test

Ebeam sterilization tests were performed on different coated staples. A focused beam of electrons was directed onto the coated staples for sterilization. The staple aging performance was evaluated by the percentage of passing legs before and after ebeam sterilization in Table 3. CSS polymer and Parylene coating are more resistant to ebeam impacts compared to PLA-PCL.

TABLE 6
Percentage of Passing Staple Legs for Coated
Staples Before and After Ebeam Sterilization

| | | | Prior to ebeam | Post ebeam |
|---|---|---|---|---|
| | | | sterilization | sterilization |
| | | | % passing | % passing |
| | | | staple legs | staple legs |
| | | | after aging | after aging |
| Polymer | Coating | Minimum | 7 days | 7 days |
| Coating | thickness | thickness | in SGF | in SGF |
| Parylene | 6 μm | 2 μm | 67 | 60 |
| CSS Polymer | 6 μm | 2 μm | 50 | 60 |
| PLA-PCL | 6 μm | 2 μm | 80 | 10 |

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

1. A coated staple comprising:
a staple 300; and
a coating comprising a copolymer comprising a residue that is a succinate-based monomer,
wherein the coating is disposed on at least a portion of a surface of the staple, and the coating has an elongation at break (ε_{break}) of at least about 11%.

2. The coated staple of claim 1, wherein the coating has a Young's modulus (E) of about 40 MPa to about 110 MPa, and/or wherein the coating has an ultimate tensile strength (UTS) of about 20 MPa to about 60 MPa.

3. The coated staple of claim 1,
wherein the coating has a glass transition temperature (Tg) of about −20° C. to about 10° C.

4. The coated staple of claim 3, wherein the coating has a glass transition temperature (Tg) of about −8° C.

5. The coated staple of claim 1,
wherein when the coating has a thickness of about 2 μm to about 22 μm, the water uptake of the coating is less than about 1.9% by weight.

6. The coated staple of claim 5, wherein the coating has a thickness of about 6 μm and/or wherein the coating wherein the water uptake of the coating is less than 1.0% by weight.

7. The coated staple of claim 1, wherein: the coating is configured to delay degradation of the at least one portion of the staple 300 in vivo; the staple 300 is absorbable; and/or the coating is absorbable.

8. The coated staple of claim 1, wherein the copolymer comprises about 10 mole % to about 40 mole % of the residue that is a succinate-based monomer.

9. The coated staple of claim 1, wherein the residue that is a succinate-based monomer is a product of a succinic acid and a hydroxyl thiol.

10. The coated staple of claim 1, wherein the succinate-based monomer has the formula of: 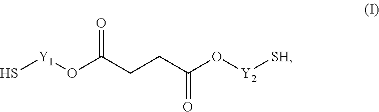
wherein Y₁ and Y₂ are each independently alkylene, ethoxy, arylene, or any combination thereof.

11. The coated staple of claim 1, wherein the copolymer further comprises a residue of C₁ to C₁₂ bis-propriolate.

12. The coated staple of claim 1, wherein the copolymer further comprises a residue of C₁ to C₁₂ bis-propriolate having the formula of: 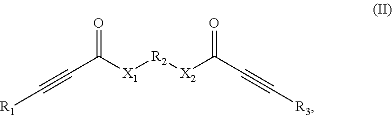
where R₁ and R³ are each independently H, alkyl, or aryl group; R₂ is a covalent bond, alkylene, ethoxy, arylene, or any combination thereof; and X₁ and X₂ are each independently O, NH, or S.

13. The coated staple of claim 1, wherein the copolymer further comprises a residue of a multi-functional thiol having a formula of HS—R₄—SH (III), wherein R⁴ is alkylene, ethoxy, arylene, or any combination thereof.

14. The coated staple of claim 1, wherein the copolymer has a formula of: 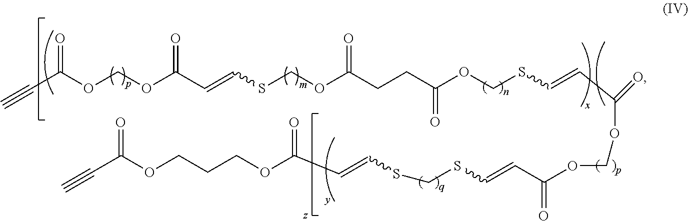
wherein each of m, n, p, and q is independently an integral of 1-12, each of x and y is independently a mole fraction between 0 and 1, the total of x and y is 1, and z is an integer from 10 to 500,
or 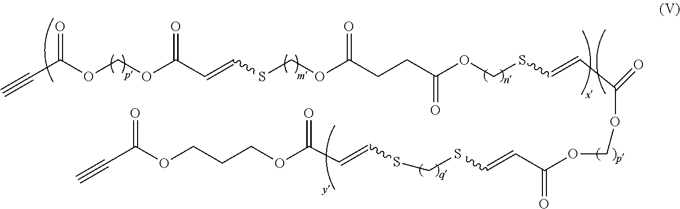
wherein each of m′, n′, p′, and q′ is independently an integral of 1-12, each of x′ and y′ is independently an integer from 10 to 500.

15. The coated staple of claim 14, wherein the copolymer has the formula of: 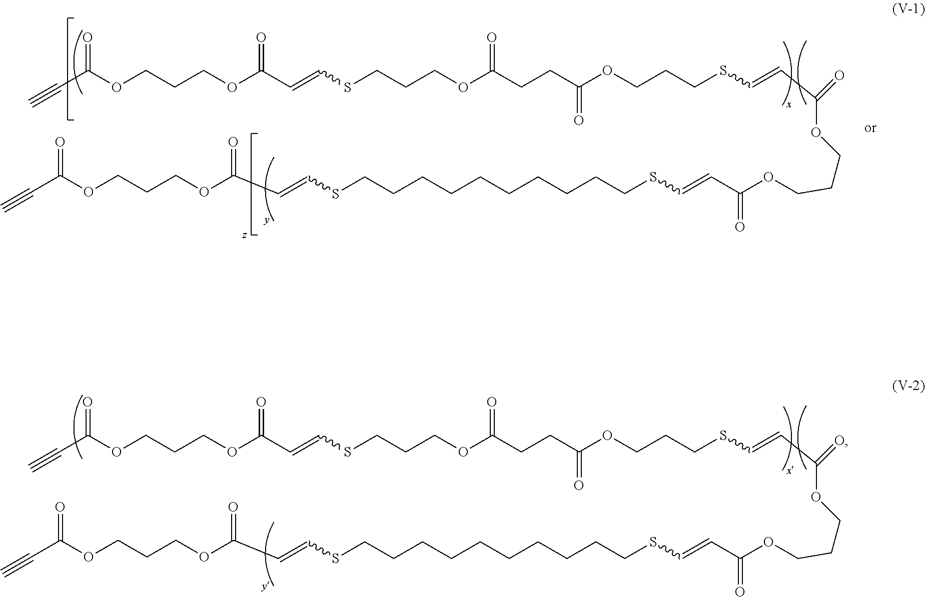
wherein x and y are mole fractions and the total of x and y is 1, z is an integer from 10 to 500, x′ and y′ are each independently an integer from 10 to 500, optionally wherein x is a mole fraction from about 0.05 to about 0.40.

16. The coated staple of claim 1, wherein:
the staple comprises an absorbable metal that is significantly, principally, or substantially composed of magnesium, optionally further comprising one or more metallic elements selected from an alkali metal, an alkaline earth metal, a transition metal, or lanthanides, wherein the one or more metallic elements selected from aluminum, beryllium, calcium, cerium, copper, gadolinium, lithium, iron, manganese, molybdenum, neodymium, nickel, silver, strontium, thorium, tin, titanium, vanadium, yttrium, zirconium, dysprosium, or zinc; or
the staple comprises an absorbable metal that is significantly, principally, or substantially composed of zinc, optionally further comprising one or more metallic elements selected from an alkali metal, an alkaline earth metal, a transition metal, or lanthanides, wherein the one or more metallic elements selected from aluminum, beryllium, calcium, cerium, copper, gadolinium, lithium, iron, manganese, molybdenum, neodymium, nickel, silver, strontium, thorium, tin, titanium, vanadium, yttrium, zirconium, dysprosium, or magnesium.

17. The coated staple of claim 1, comprising a crown 1002, a first leg 1004, and a second leg 1006, a radius of curvature 1008 between the crown 1002 and each of the first leg 1004 and the second leg 1006 being greater than or equal to 0.005 inches.

18. A method of making the coated staple of claim 1, comprising:
coating at least a portion of a surface of the staple with the copolymer comprising a residue that is a succinate monomer, wherein coating is conducted via electrospinning, electrospray, dip coating, thermal spray, screen printing, an extrusion process comprising pultrusion, direct deposition, laser sintering, overmolding, spray coating, chemical vapor deposition, reel-to-reel coating, or combinations thereof.

19. A surgical device 100, comprising:
a cartridge body 200, comprising:
a deck; and
a staple cavity defined in the deck, the staple cavity comprising the coated staple of claim 1.

20. The surgical device 100 of claim 19 further comprising:
an end effector 106 comprising:
a first jaw; and
a second jaw, wherein at least one of said first jaw and said second jaw is movable between an open position and a clamped position, and wherein the cartridge body 200 is positionable within the second jaw.
